(19) United States
(12) Patent Application Publication
(10) Pub. No.: US 2026/0306662 A1
(43) Pub. Date: Oct. 8, 2026

(54) MOLECULAR-GENETIC STRATEGY FOR THE REACTIVATION OF JUVENILE PLASTICITY IN TARGETED ADULT NEURAL CIRCUITS

(71) Applicant: The Regents of the University of California, Oakland, CA (US)

(72) Inventors: Sunil Gandhi, Irvine, CA (US); Taylor Nakayama, Irvine, CA (US)

(21) Appl. No.: 19/480,946

(22) PCT Filed: May 3, 2024
(86) PCT No.: PCT/US2024/027871

§ 371 Date: Nov. 3, 2025

Related U.S. Application Data
(60) Provisional application No. 63/499,826, filed on May 3, 2023.

(51) Int. Cl. A61K 48/00; C12N 15/86; G01N 33/50

(52) U.S. Cl. CPC A61K 48/0033; C12N 15/86; G01N 33/502; C12N 2750/14143

(57) ABSTRACT

Transplantation of inhibitory neuron progenitors rekindles a targeted, time-limited phase of critical period plasticity within recipient brain areas. This restoration of youthful plasticity establishes a therapeutic environment conducive to circuit reorganization, consistently demonstrated to ameliorate cognitive and functional impairments. Remarkably, the mere overexpression of Calb1 in adult inhibitory neurons via viral vectors is adequate to reactivate critical period plasticity without the need for transplantation. Consequently, methodologies and compositions for augmenting Calb1 expression as a therapeutic avenue to reactivate plasticity are described herein.

### CROSS-REFERENCE TO RELATED APPLICATIONS

This application claims benefit of U.S. Provisional Application No. 63/499,826 filed May 3, 2023, the specification of which is incorporated herein in its entirety by reference.

### STATEMENT REGARDING FEDERALLY SPONSORED RESEARCH OR DEVELOPMENT

This invention was made with government support under Grant Nos. 1F31EY034032-01 and R01EY029490 awarded by National Institutes of Health. The government has certain rights in the invention.

### FIELD OF THE INVENTION

The present invention features methodologies and compositions for modulating Calb1 expression as a therapeutic avenue to reactivate plasticity.

### BACKGROUND OF THE INVENTION

Critical periods are well-defined windows during postnatal development in which neural circuits are particularly receptive to sensory input. This enhanced state of plasticity enables experience to powerfully rearrange and optimize local wiring for concurrent conditions. Unlike transient alterations enabled by adult forms of plasticity, changes occurring within critical periods are strongly cemented and, therefore, regulate the circuit's functional capacity well into adulthood. While critical period timing has been strongly linked to the maturation of inhibitory circuits, it has been challenging to separate mechanisms specific to plasticity from those specific for development. Faithful reactivation of critical period plasticity is likely mediated by factors expressed during early stages in inhibitory neuron development.

The brain's inhibitory neuron progenitors originate from the ganglionic eminence, a transient embryonic brain structure whose subregions give rise to different inhibitory subpopulations with varying trajectories: 1) medial ganglionic eminence (MGE), which produces cortical parvalbumin (PV) and somatostatin (SOM) inhibitory neurons, 2) lateral ganglionic eminence (LGE), which produces striatal PV and SOM inhibitory neurons, and 3) caudal ganglionic eminence (CGE), which produces mostly cortical vasoactive intestinal polypeptide (VIP) inhibitory neurons. Due to their strong retention of developmental programming, transplanted inhibitory neuron progenitors mature appropriately and form synergistic connections with host circuitry. Unlike LGE and CGE, the transplantation of MGE-derived inhibitory neuron progenitors powerfully reactivates critical period plasticity within recipient brain regions. Notably, the reactivated critical period does not occur immediately but nearly a month after transplantation-the original timing of the transplanted inhibitory neurons' maturation. This alignment in temporal programming strongly suggests that the transplant-reactivated critical period is triggered by factors intrinsic to the transplanted cells.

Given its incredible capacity for correcting dysfunctional circuitry, inhibitory neuron transplantation has gamered positive attention from a broad range of CNS disease/disorders such as epilepsy, autism, stroke, schizophrenia, Alzheimer's, Parkinson's, and neuropathic pain. Though many of these involve imbalances in excitation/inhibition, pharmacological enhancements of inhibition fail to fully phenocopy transplant-mediated functional recovery. Consistent with observations from the juvenile critical period, inhibitory neuron transplantation likely engages mechanisms beyond GABA transmission to rescue deficits. Despite extensive application, the cell and molecular mechanisms underlying inhibitory neuron transplantation remain unclear. Furthermore, candidate molecules that have been previously implicated in juvenile critical period plasticity insufficiently explain, or have not been examined in the context of, transplant-reactivated critical periods.

### BRIEF SUMMARY OF THE INVENTION

It is an objective of the present invention to provide compositions and methods that allow for the reactivation of plasticity without reliance on a cellular vehicle, as specified in the independent claims. Embodiments of the invention are given in the dependent claims. Embodiments of the present invention can be freely combined with each other if they are not mutually exclusive.

In some embodiments, the present invention features a method of increasing neural plasticity (e.g., experience-dependent plasticity) in a subject in need thereof. The method may comprise administering a gene therapy agent to the subject, said gene therapy agent configured to modulate (e.g., increase) the expression of calbindin (Calb1) in a population of neuronal cells in the subject. In some embodiments, modulating (e.g., increasing) the expression of Calb1 increases neural plasticity (e.g., experience-dependent plasticity) in the subject. In other embodiments, modulating (e.g., increasing) the expression of Calb1 increases neural plasticity (e.g., experience-dependent plasticity) in a population of neuronal cells in the subject. Non-limiting examples of gene therapy agents include but are not limited to viral vectors, exosomes, nanoparticles, small interfering RNAs (siRNAs) and microRNAs (miRNAs), antisense oligonucleotides (ASOs), aptamers, modified cells, or the like. In some embodiments, the population of neuronal cells comprises excitatory neurons, inhibitory neurons, or a combination thereof. In some embodiments, the gene therapy agent directly increases the expression of Calb1. In other embodiments, the gene therapy agent indirectly increases the expression of Calb1. In certain embodiments. the method may further comprise measuring the expression of Calb1 prior to administering the gene therapy agent.

In other embodiments, the present invention features a method of treating a CNS disease or disorder in a subject in need thereof. The method may comprise administering a gene therapy agent to the subject, said gene therapy agent configured to modulate (e.g., increase) the expression of calbindin (Calb1) in a population of neuronal cells in the subject. In some embodiments, modulating (e.g., increasing) the expression of Calb1 increases neural plasticity (e.g., experience-dependent plasticity) in the subject, thus facilitating the treatment or regression of the CNS disease or disorder. In other embodiments, modulating (e.g., increasing) the expression of Calb1 increases neural plasticity (e.g., experience-dependent plasticity) a population of neuronal cells in the subject, thus facilitating the treatment or regression of the CNS disease or disorder. In some embodiments, the CNS disorder or diseases may include epilepsy, autism, stroke, schizophrenia, Alzheimer's, Parkinson's, or neuropathic pain. Non-limiting examples of gene therapy agents include but are not limited to viral vectors, exosomes, nanoparticles, small interfering RNAs (siRNAs) and microRNAs (miRNAs), antisense oligonucleotides (ASOs), aptamers, modified cells, or the like. In some embodiments, the population of neuronal cells comprises excitatory neurons, inhibitory neurons, or a combination thereof. In some embodiments, the gene therapy agent directly increases the expression of Calb1. In other embodiments, the gene therapy agent indirectly increases the expression of Calb1. In certain embodiments, the method may further comprise measuring the expression of Calb1 prior to administering the gene therapy agent.

In certain embodiments, the methods described herein may be implemented within specific adult neural circuits.

In some embodiments, the present invention features a method of screening for experience-dependent plasticity-inducing factors and agents. The method may comprises administering (or having administered) one or more factors and agents to the sample and measuring (or having measured) a level expression of calbindin (Calb1). In other embodiments, the method comprises obtaining (or having obtained) a sample, administering (or having administered) one or more factors and agents to the sample and measuring (or having measured) a level expression of calbindin (Calb1). In some embodiments, an increase in the expression of Calb1 indicates the one or more factors and agents induce experience-dependent plasticity. In some embodiments, the sample is a biological sample (e.g., ex vivo tissue, primary cultures, IPSCs, organoids, or the like).

In other embodiments, the present invention features a method of screening for experience-dependent plasticity-inducing factors and agents. The method may comprise administering (or having administered) one or more factors and agents to a subject or suitable animal model and measuring (or having administered) a level expression of calbindin (Calb1). In some embodiments, an increase in the expression of Calb1 indicates the one or more factors and agents induce experience-dependent plasticity. In certain embodiments, the method may further comprise measuring (or having measured) the level of expression of calbindin prior to administering the one or more factors and agents.

One of the unique and inventive technical features of the present invention is the targeting of a singular factor, Calb1, to mimic transplant-induced plasticity. Without wishing to limit the invention to any theory or mechanism, it is believed that the technical feature of the present invention advantageously provides for therapies that do not rely on a cellular vehicle. None of the presently known prior references or works have the unique inventive technical feature of the present invention.

Moreover, the prior references teach away from the present invention. For instance, the utilization of embryonic donor tissue renders direct clinical translation of inhibitory neuron transplantation impractical. Additionally, xenotransplantation necessitates continual immunosuppression, while existing protocols for iPSC-derived inhibitory neuron progenitors encounter hurdles concerning yield and purity. Irrespective of their origin, preclinical studies on inhibitory neuron transplantation highlight the susceptibility of immature transplanted cells to active pathology. Hence, the present invention signifies a notable progression, employing a singular factor, Calb1, to mimic transplant-induced plasticity without reliance on a cellular vehicle. Consequently, the therapies outlined herein offer enhanced safety and control.

In addition, techniques aimed at enhancing general plasticity, such as social enrichment, exercise, or exposure to novelty, may only yield temporary effects and are constrained by their capacity to induce significant changes. Moreover, strategies to augment experience-dependent plasticity in adulthood, such as reducing inhibitory mechanisms or removing natural brakes on juvenile plasticity, do not always translate seamlessly. For instance, diminishing inhibition may increase susceptibility to seizures, while eliminating plasticity brakes can impede memory consolidation.

Furthermore, the inventive technical features of the present invention contributed to a surprising result. For example, the Inventors surprisingly found that increasing the expression of a singular factor, i.e., Calb1, in a subset of neurons results in a significant circuit-wide impact. Notably, increasing Calb1 expression in adulthood, particularly within inhibitory neurons, yields a beneficial effect on plasticity and can replicate the transplantation effect, even in the absence of additional factors or cells.

Moreover, it is noteworthy that LGE transplantation, unexpectedly, does not elicit any discernible impact on Calb1 expression levels beyond those observed in non-transplanted adults. The noticeable increase in Calb1 expression remains distinctly associated with MGE transplantation, aligning consistently with functional analyses conducted in previous transplantation studies.

Any feature or combination of features described herein are included within the scope of the present invention provided that the features included in any such combination are not mutually inconsistent as will be apparent from the context, this specification, and the knowledge of one of ordinary skills in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which

FIGS. 1A and 1B show the experimental methods used to obtain the data described herein. Donor mice provided embryos from which GABAergic interneurons were isolated, which were then injected into adult recipients (hosts) with ZsGreen-labeled host interneurons. Inventors have established that donor MGE cells induce a new critical period for experience-dependent plasticity in the host animal 35 days after transplantation (Southwell, 2010; Davis, 2015; Zheng, 2021). ZsGreen-labeled host interneurons were sorted out and sequenced. Differential expression analysis was performed between host interneurons from MGE transplant, LGE transplant, and non-transplanted mice during the induced critical period (35 DAT) after closure (70 DAT). FIG. 1B shows a schematic detailing the donor tissue source, cells produced from those dissections, and the effect of each transplant type.

FIGS. 2A, 2B, and 2C show validation that calbindin protein levels are increasing and that the increase is due to MGE transplantation specifically. Transplant-induced increases in adult Calbindin expression are similar to expression levels seen during the peak of juvenile experience-dependent plasticity. FIG. 2B shows the quantification of Calb1 protein and how expression levels change with age (from p28 to adult no-transplant) and transplantation groups (adult no-transplant compared to MGE and LGE recipients). FIG. 2C shows that the increase in Calbindin protein expression is not due to differences in the number of inhibitory neurons between groups.

FIGS. 3A, 3B, and 3C shows a schematic of postnatal development detailing the transient window of experience-dependent plasticity. Key timepoints during visual development are labeled. FIG. 3B shows that during normal development in the mouse visual cortex, there is an increase and subsequent decrease in calbindin levels that aligns with the peak of experience-dependent plasticity (increase) and immediate closure of that plasticity window (decrease). This provides a mechanistic rationale for why induced increases in calbindin levels can recapitulate experience-dependent plasticity. FIG. 3C shows quantification of Calb1 protein in cortical layers across age and transplantation groups, and in particular, an increase in Calb1 deep layer expression following MGE transplantation.

FIGS. 4A, 4B, 4C, 4D, 4E, 4F, and 4G shows shows experimental setup of viral injection to overexpress Calb1 in endogenous GABAergic interneurons and imaging to determine changes in circuit plasticity following monocular deprivation. FIG. 4B shows the successful, focal expression of Calb1 virus in mouse visual cortex. FIG. 4C shows quantification of a successful increase in Calb1 protein after viral overexpression. FIGS. 4D and 4E shows physiological maps of changes in eye-specific responses before and after monocular deprivation between virus recipients and saline-injected controls. FIG. 4F shows induction of experience-dependent plasticity specific to Calb1 overexpression vs. saline-injected controls. The extent of plasticity is similar to levels seen during the peak of juvenile experience-dependent plasticity. FIG. 4G further shows that the experience-dependent plasticity induced by Calb1 overexpression is mediated by a decrease in contra response (a well-established feature of experience-dependent plasticity).

### DETAILED DESCRIPTION OF THE INVENTION

Disclosed are various peptides, solvents, solutions, carriers, and/or components to be used to prepare compositions to be used within the methods disclosed herein. Also disclosed are the various steps, elements, amounts, routes of administration, symptoms, and/or treatments that are used or observed when performing the disclosed methods, as well as the methods themselves. These and other materials, steps, and/or elements are disclosed herein, and it is understood that when combinations, subsets, interactions, groups, etc. of these materials are disclosed, while specific reference of each various individual and collective combination and permutation of these compounds may not be explicitly disclosed, each is specifically contemplated and described herein. It is also to be understood that the terminology used herein is for the purpose of describing particular embodiments only and is not intended to be limiting.

Unless otherwise explained, all technical and scientific terms used herein have the same meaning as commonly understood by one of ordinary skill in the art to which a disclosed invention belongs. The singular terms “a,” “an,” and “the” include plural referents unless context clearly indicates otherwise. Similarly, the word “or” is intended to include “and” unless the context clearly indicates otherwise. The term “comprising” means that other elements can also be present in addition to the defined elements presented. The use of “comprising” indicates inclusion rather than limitation. Stated another way, the term “comprising” means “including principally, but not necessary solely”. Furthermore, variation of the word “comprising”, such as “comprise” and “comprises”, have correspondingly the same meanings. In one respect, the technology described herein related to the herein described compositions, methods, and respective component(s) thereof, as essential to the invention, yet open to the inclusion of unspecified elements, essential or not (“comprising”).

Suitable methods and materials for the practice and/or testing of embodiments of the disclosure are described below. Such methods and materials are illustrative only and are not intended to be limiting. Other methods and materials similar or equivalent to those described herein can be used. For example, conventional methods well known in the art to which the disclosure pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999, Gene Expression Technology (Methods in Enzymology, Vol. 185, edited by D. Goeddel, 1991. Academic Press, San Diego, Calif), “Guide to Protein Purification” in Methods in Enzymology (M. P. Deutshcer, ed., (1990) Academic Press, Inc.): PCR Protocols: A Guide to Methods and Applications (Innis, et al. 1990. Academic Press, San Diego, Calif.), Culture of Animal Cells: A Manual of Basic Technique, 2nd Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.), the disclosures of which are incorporated in their entirety herein by reference.

All publications, patent applications, patents, and other references mentioned herein are incorporated by reference in their entirety for all purposes. In case of conflict, the present specification, including explanations of terms, will control.

Although methods and materials similar or equivalent to those described herein can be used to practice or test the disclosed technology, suitable methods and materials are described below. The materials, methods, and examples are illustrative only and not intended to be limiting.

A “subject” is an individual and includes, but is not limited to, a mammal (e.g., a human, horse, pig, rabbit, dog, sheep, goat, non-human primate, cow, cat, guinea pig, or rodent), a fish, a bird, a reptile or an amphibian. The term does not denote a particular age or sex. Thus, adult and newborn subjects, as well as fetuses, whether male or female, are intended to be included. A “patient” is a subject afflicted with a disease or disorder. The term “patient” includes human and veterinary subjects.

As used herein, the terms “treat,” “treating,” or “treatment” refer to both therapeutic treatment and prophylactic or preventative measures, with the objective of preventing, reducing, slowing down (lessen), inhibiting, or eliminating an undesired physiological change, symptom, disease, or disorder. For example, the disease may be a CNS disease. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented or onset delayed. Optionally, the subject or patient may be identified (e.g., diagnosed) as one suffering from the disease or condition prior to administration of the compositions of the invention. Subjects at risk for the disease can be identified by, for example, any or a combination of appropriate diagnostic or prognostic assays known in the art.

As used herein, “clinical improvement” may refer to a noticeable reduction in the symptoms of a disorder, or cessation thereof.

The terms “manage,” “managing,” and “management” refer to preventing or slowing the progression, spread or worsening of a disease or disorder, or of one or more symptoms thereof. In certain cases, the beneficial effects that a subject derives from a prophylactic or therapeutic agent do not result in a cure of the disease or disorder.

The terms “regress,” “regressing,” and “regression” may refer to a decrease in the size of a tumor or in the extent of cancer in the body. In some embodiments, “regression” may refer to a decrease in severity of the disease and/or decrease in the size of a tumor. In some embodiments, regression may generally refer to lighter symptoms without the disease completely disappearing. In certain cases, the beneficial effects that a subject derives from a prophylactic or therapeutic agent do not result in a cure of the disease or disorder. In some embodiments, symptoms of the disease may return.

The terms “administering” and “administration” refer to methods of providing a pharmaceutical preparation, composition, or formulation to a subject. The compositions described herein can be administered in a number of ways depending on whether local or systemic treatment is desired, and on the area to be treated. Such methods are well known to those skilled in the art and include, but are not limited to, administering the compositions orally, intranasally, parenterally (e.g., intravenously and subcutaneously), by intramuscular injection, by intraperitoneal injection, intrathecally, transdermally, extracorporeally, topically or the like.

As used herein, the term “neural plasticity” refers to a broad descriptor encapsulating the brain's receptivity to change its function or connections.

As used herein, the term “experience-dependent plasticity” is a specific term that refers to the brain's receptivity to change instructed by external cues (e.g., sensory signals/stimuli). Normally restricted to early postnatal development and with extensive capacity for change. The resulting changes are strongly cemented.

As used herein, the term “juvenile plastic state” or “juvenile plasticity” (e.g., experience-dependent plasticity) may be used interchangeably and refer to an increased responsiveness of targeted neurons to incoming signals, similar to responses seen during development (e.g., during early postnatal development).

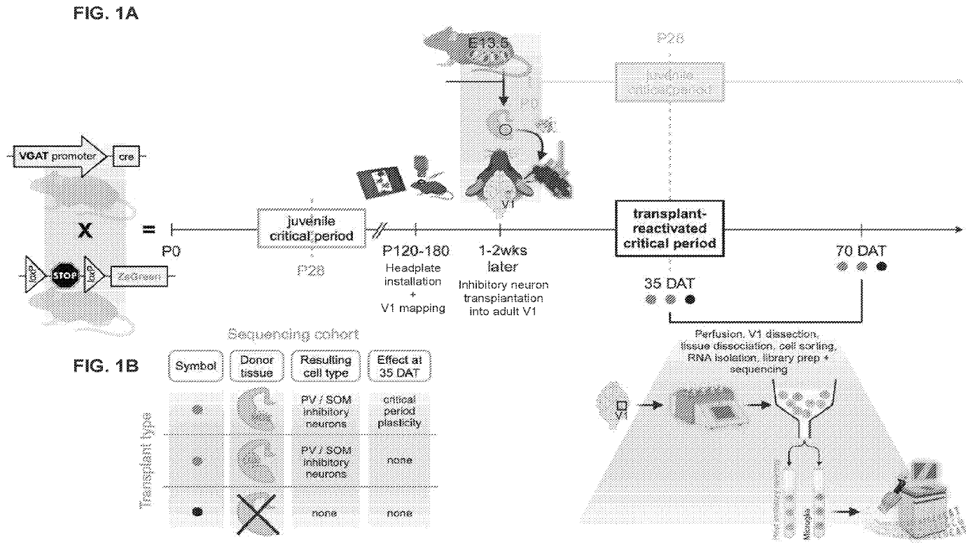

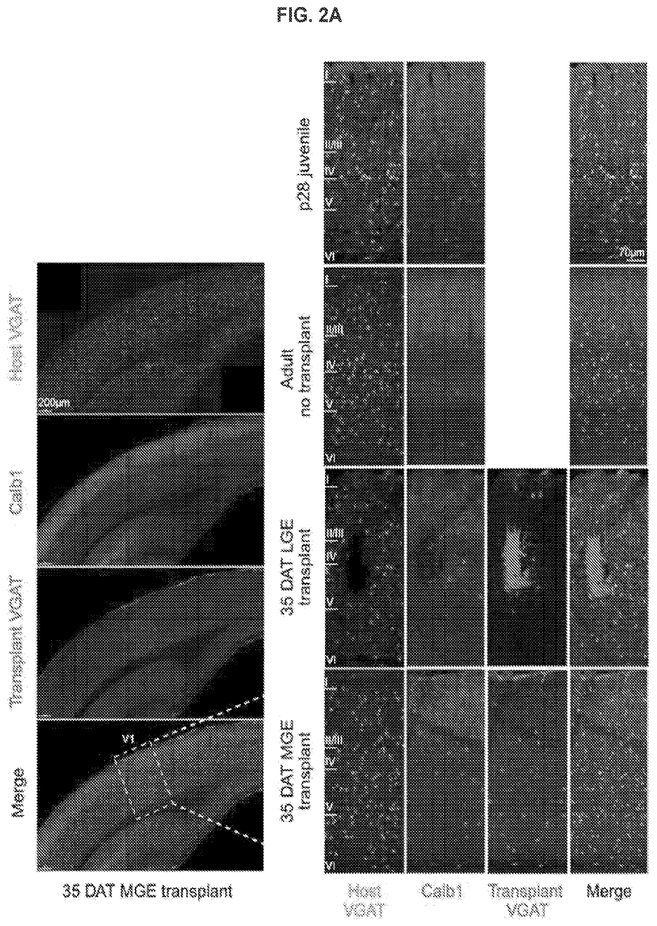

Referring now to FIGS. 1A-4G, the present invention features compositions and methods that allow for the reactivation of transplant-induced plasticity without reliance on a cellular vehicle.

The present invention features a method of increasing neural plasticity (e.g., experience-dependent plasticity) in a subject (e.g., in need thereof). The method may comprise increasing the expression of calbindin (Calb1) in the subject's neuronal cells (e.g., excitatory neurons, inhibitory neurons, or a combination thereof). In some embodiments, the method comprises increasing the expression of Calb1 in a portion of the subject's neuronal cells. In some embodiments, increasing the expression of calbindin increases the level of neural plasticity in the subject. In other embodiments, increasing the expression of calbindin increases a level of experience-dependent plasticity in the subject.

In other embodiments, the method comprises increasing expression of calbindin in a population of the subject's neuronal cells (e.g., excitatory neurons or inhibitory neurons). In some embodiments, increasing the expression of calbindin increases the level of neural plasticity in the subject's neuronal cells. In other embodiments, increasing the expression of calbindin increases the level of neural plasticity in a population of the subject's neuronal cells (e.g., excitatory neurons or inhibitory neurons). In some embodiments, increasing the expression of calbindin increases the level of experience-dependent plasticity in the subject's neuronal cells. In other embodiments, increasing the expression of calbindin increases the level of experience-dependent plasticity in a population of the subject's neuronal cells (e.g., excitatory neurons or inhibitory neurons).

In certain embodiments, the assessment of neural plasticity involves employing a range of well-established neurophysiological, behavioral, and histological assays in the art. One such assay highlighted herein is ocular dominance plasticity, originally identified by Hubel and Wiesel. This assay characterizes a specific critical period during early postnatal development, wherein a temporary visual disparity between the two eyes induces a lasting modification in neurophysiological responses within the visual system, favoring the dominant eye. In some embodiments, the methods utilized herein may include evaluating responses from each subject's individual eyes both before (e.g., an adult subject baseline) and after the temporary visual disparity, as described earlier. A discernible alteration in responses indicates the presence of experience-dependent plasticity (e.g., successful reactivation), while the absence of a significant shift suggests no changes in plasticity relative to the adult baseline. However, the present invention extends beyond the aforementioned methods for measuring plasticity and may encompass existing region-specific, disease-specific, or other relevant methodologies. Following a similar protocol, baseline measurements may be compared with the effects observed after the administration of a composition (e.g., small molecule or a gene therapy agent both configured to increase Calb1).

In some embodiments, the population of neuronal cells (e.g., neurons) comprises inhibitory neurons or interneurons. In other embodiments, the population of neuronal cells (e.g., neurons) comprises excitatory neurons, inhibitory neurons, or a combination thereof.

In accordance with the present invention, increasing Calb1 expression could be accomplished through a diverse array of properly engineered delivery methods. These methods include but are not limited to the use of viral vectors, exosomes, nanoparticles, miRNA/siRNA, ASOs, aptamers, modified cells, and others. These delivery systems can transport DNA, RNA, factors, or their derivatives, directly or indirectly facilitating the upregulation of Calb1 expression.

In some embodiments, increases in Calb1 expression can be assessed through various techniques, including RNA/DNA analysis (such as northern blot, real-time quantitative PCR, sequencing), protein analysis (such as western blot, immunohistochemistry), and among others. In addition, it should be noted that the present invention is not limited to the methods disclosed herein, and any method known in the art may be employed.

The present invention features a method of increasing neural plasticity (e.g., experience-dependent plasticity) in a subject (e.g., in need thereof). The method may comprise administering a composition (e.g., a composition comprising a small molecule or a gene therapy agent) that increases the expression of calbindin (Calb1) to the subject's neuronal cells (e.g., excitatory neurons, inhibitory neurons, or a combination thereof). In some embodiments, the method comprises administering a composition (e.g., a composition comprising a small molecule or a gene therapy agent) that increases the expression of Calb1 to a portion of the subject's neuronal cells (e.g., to a population of neuronal cells). In some embodiments, increasing the expression of calbindin increases the level of neural plasticity in the subject. In other embodiments, increasing the expression of calbindin increases a level of experience-dependent plasticity in the subject.

In certain embodiments, the method described herein may additionally comprise assessing the baseline expression of Calb1 in the targeted region/cells before administering the composition. Furthermore, an elevation in Calb1 expression can be determined by comparing the baseline level of Calb1 expression with its expression subsequent to the administration of the composition.

In certain embodiments, the composition directly increases the expression of Calb1. In other embodiments, the composition may indirectly facilitate the increase in Calb1 expression. In some embodiments, compositions described herein may comprise one or a combination of small molecules, peptides, enzymes, antibodies, oligonucleotides, drugs, or the like.

In some embodiments, the present invention features a method of increasing neural plasticity (e.g., experience-dependent plasticity) in a subject in need thereof. The method may comprise administering a composition to the subject, said composition configured to modulate (e.g., increase) the expression of calbindin (Calb1) in a population of neuronal cells in the subject. In some embodiments, modulating (e.g., increasing) the expression of Calb1 increases neural plasticity (e.g., experience-dependent plasticity) in the subject. In other embodiments, modulating (e.g., increasing) the expression of Calb1 increases neural plasticity (e.g., experience-dependent plasticity) in a population of neuronal cells in the subject. In some embodiments, compositions described herein may comprise one or a combination of small molecules, peptides, enzymes, antibodies, oligonucleotides, drugs, or the like. In some embodiments, the population of neuronal cells comprises excitatory neurons, inhibitory neurons, or a combination thereof. In certain embodiments, the method may further comprise measuring the expression of Calb1 prior to administering the composition (e.g., comprising a small molecule).

In other embodiments, the present invention features a method of treating a CNS disease or disorder in a subject in need thereof. The method may comprise administering a composition to the subject, said composition configured to modulate (e.g., increase) the expression of calbindin (Calb1) in a population of neuronal cells in the subject. In some embodiments, modulating (e.g., increasing) the expression of Calb1 increases neural plasticity (e.g., experience-dependent plasticity) in the subject, thus facilitating the treatment or regression of the CNS disease or disorder. In other embodiments, modulating (e.g., increasing) the expression of Calb1 increases neural plasticity (e.g., experience-dependent plasticity) a population of neuronal cells in the subject, thus facilitating the treatment or regression of the CNS disease or disorder. In some embodiments, the CNS disorder or diseases may include epilepsy, autism, stroke, schizophrenia, Alzheimer's, Parkinson's, or neuropathic pain. In some embodiments, compositions described herein may comprise one or a combination of small molecules, peptides, enzymes, antibodies, oligonucleotides, drugs, or the like. In certain embodiments, the method may further comprise measuring the expression of Calb1 prior to administering the composition.

The present invention may also feature a method of increasing neural plasticity (e.g., experience-dependent plasticity) in a subject in need thereof. In some embodiments, the method comprises administering a gene therapy agent to the subject; said gene therapy agent is configured to increase the expression of calbindin in a population of the subject's neuronal cells, wherein increasing the expression of calbindin increases a level of experience-dependent plasticity in the subject.

In certain embodiments, the gene therapy agent directly increases the expression of Calb1. For example, a vector incorporating a Calb1 sequence, specifically designed to boost Calb1 expression, may be utilized. In other embodiments, the gene therapy agent may indirectly facilitate the increase in Calb1 expression. This can be achieved, for example, through a vector containing regulatory RNA/gene sequences known to modulate Calb1 expression. Such regulation may involve inducing the cell to increase Calbi expression or preventing age/disease-related decline in Calb1 expression by containing sequences of regulatory RNA/gene known to decrease Calb1 expression. In some embodiments, gene therapy agents may include but are not limited to viral vectors, exosomes, nanoparticles, small interfering RNA (siRNA) and microRNA (miRNA), antisense oligonucleotide (ASO), aptamers, modified cells, or the like.

In some embodiments, the method comprises administering a viral vector to the subject, said viral vector configured to increase the expression of calbindin in a population of the subject's neuronal cells (e.g. inhibitory neurons, excitatory neurons, or a combination thereof). In some embodiments, increasing the expression of calbindin increases a level of experience-dependent plasticity in the subject. Non-limiting examples of viral vectors may include adeno-associated viruses (AAVs), adenoviruses, lentiviruses, or the like.

In alternative embodiments, the method comprises administering exosomes (e.g., comprising a therapeutic agent) to the subject, said exosome configured to increase the expression of calbindin in a population of the subject's neuronal cells (e.g., inhibitory neurons, excitatory neurons, or a combination thereof). In some embodiments, increasing the expression of calbindin increases a level of experience-dependent plasticity in the subject. In accordance with the present invention, exosomes represent extracellular vesicles capable of being engineered to transport therapeutic molecules, such as the Calb1 sequence, into surrounding cells upon uptake.

In other embodiments, the method comprises administering nanoparticles (e.g., comprising a therapeutic agent) to the subject, said nanoparticles configured to increase the expression of calbindin in a population of the subject's neuronal cells (e.g., inhibitory neurons, excitatory neurons, or a combination thereof). In some embodiments, increasing the expression of calbindin increases a level of experience-dependent plasticity in the subject. In some embodiments, therapeutic molecules like Calb1 may be encapsulated within nanoparticles such as lipids or metals, facilitating their delivery into targeted cells.

In certain embodiments, methods described herein may target neurons that reside in disease-affected/dysregulated regions of a subject's brain.

In some embodiments, methods described herein may be used to treat CNS disease/disorders such as epilepsy, autism, stroke, schizophrenia, Alzheimer's, Parkinson's, and neuropathic pain.

In certain embodiments, the method described herein may additionally comprise assessing the baseline expression of Calb1 in the targeted region/cells before administering the gene therapy agent. Furthermore, an elevation in Calb1 expression can be determined by comparing the baseline level of Calb1 expression with its expression subsequent to the administration of the gene therapy agents.

The methods for enhancing plasticity, such as experience-dependent plasticity, described herein may benefit a wide spectrum of diseases and dysregulations within brain circuits. For example, the induction of plasticity via Calb1 may find application in various conditions, including but not limited to epilepsy, amblyopia, stroke, schizophreniform disorders, Alzheimer's disease, Parkinson's disease, and neuropathic pain. Furthermore, these methods could complement existing neurotherapeutics by priming the neuronal and brain circuitry to be more receptive, thereby potentially enhancing the efficacy of subsequent treatments.

In some embodiments, the present invention may feature a method of treating a central nervous system (CNS) disease or disorder. The method may comprise administering (e.g., contacting) a gene therapy agent that increases the expression of calbindin (Calb1) to the neuronal cells (e.g., excitatory neurons, inhibitory neurons, or a combination thereof) in the subject. In some embodiments, the method comprises administering (e.g., contacting) a gene therapy agent that increases the expression of Calb1 to a portion of the subject's neuronal cells (e.g., to a population of neuronal cells). In some embodiments, the CNS disorder or diseases may include epilepsy, autism, stroke, schizophrenia, Alzheimer's, Parkinson's, or neuropathic pain.

In certain embodiments, the present invention features a gene therapy agent for use in the treatment of CNS diseases or disorders. The gene therapy agent, in certain embodiments, increases the expression of Calb1 in neuronal cells (e.g., excitatory neurons, inhibitory neurons, or a combination thereof) within a subject. In some embodiments, increasing the expression of Calb1 in this manner increases neural plasticity in the subject, thereby contributing to the treatment or regression of the CNS disease or disorder. Additionally, in alternative embodiments, the upregulation of Calb1 expression increases experience-dependent plasticity in the subject, further facilitating the treatment or regression of the CNS disease or disorder In some embodiments, the CNS disorder or diseases may include epilepsy, autism, stroke, schizophrenia, Alzheimer's, Parkinson's, or neuropathic pain.

In certain embodiments, the methods described herein may be implemented within specific adult neural circuits.

The present invention may further include a method for screening experience-dependent plasticity-inducing factors and agents. In certain embodiments, the method comprises increasing the expression of calbindin in a targeted population of the subject's neuronal cells. For example, after increasing Calb1 expression, targeted cells may be analyzed (e.g., sequenced) to identify recurring, strongly co-expressed factors or agents. These factors are likely to be co-regulated with Calb1 expression, and modulating their expression may enhance induced plasticity, neuronal function, survival, etc.

Moreover, the present invention may encompass a method for screening experience-dependent plasticity-inducing factors and agents. In certain embodiments, the method comprises obtaining a sample (e.g., biological sample, such as ex vivo tissue, primary cultures, IPSCs, organoids, etc.) and administering potential experience-dependent plasticity-inducing factors and agents to the sample. An elevation in calbindin expression within the sample serves as an indicator of a factor or agent capable of inducing experience-dependent plasticity. For example, in vitro settings enable researchers to screen comprehensive drug libraries and ascertain whether targeted cell cultures exhibit increased expression of Calb1, among other metrics.

In other embodiments, the method may comprise administering potential experience-dependent plasticity-inducing factors and agents to a subject (e.g., a suitable animal model, e.g., a mouse model). In some embodiments, an increase in the expression of calbindin indicates a factor or agent that induces experience-dependent plasticity. For example, a personalized treatment or reagent may be administered to a subject, and target cells may subsequently be evaluated for an increase in Calb1 expression (e.g., as a rapid indicator of successful ‘rejuvenation’).

Moreover, in additional embodiments, screening methods for experience-dependent plasticity-inducing factors and agents may include the utilization of in silico techniques, such as computational modeling.

Screening methods described herein may encompass the reassessment of existing FDA-approved or preclinical treatments, devices, etc., for their effect on Calb1 expression. Additionally, the screening process may involve testing novel gene candidates implicated in experience-dependent plasticity, utilizing increased Calb1 expression as an initial indicator In some embodiments, various combinations of these approaches may also be employed.

In some embodiments, the present invention features a method of increasing neural plasticity in a subject in need thereof. The method may comprise transplanting a population of medial ganglionic eminence (MGE) cells into the subject's neuronal tissue, wherein said transplantation increases the expression of calbindin in a population of the subject's neuronal cells, wherein increasing the expression of calbindin increases a level of experience-dependent plasticity in the subject.

In other embodiments, the present invention features a method of increasing neural plasticity in the central nervous system of an adult subject in need thereof, the method comprising transplanting a population of medial ganglionic eminence (MGE) GABAergic interneurons into the subject's neuronal tissue, wherein said transplantation increases the expression of calbindin in a population of the subject's neuronal cell.

### EXAMPLE 1

The following is a non-limiting example of the present invention. It is to be understood that said example is not intended to limit the present invention in any way. Equivalents or substitutes are within the scope of the present invention.

Animals: To label host inhibitory neurons, adult mice with green fluorescent inhibitory neurons were generated by crossing homozygous VGAT-cre females (VGAT-cre, JAX 028862) with cre-dependent ZsGreen males (AI6, JAX 007906). Resulting litters were aged to P120-P180 (4-6 mo) prior to experimental onset. To distinguish transplanted cells from endogenous host cells, donor embryos with red fluorescent inhibitory neurons were generated by crossing homozygous VGAT-cre females with cre-dependent tdTomato males (Ai14, JAX 007914). Viral injections were conducted in hemizygous VGAT-tdTomato recipient mice. Colony genotypes were periodically confirmed by PCR (Transnetyx), and successful cre recombination was individually confirmed before each experiment via fluorescence. All transplant recipients were single-housed after headplate installation. All experimental cohorts included near-equal numbers of male and female mice.

Headplates: To ensure stability for downstream intracerebral procedures, custom-printed headplates were affixed to the skulls of adult recipient mice (P120-P180). Mice received a subcutaneous injection of analgesic Carprofen (0.8 mg/ml; Rimadyl), supplemental hydration (Lactated Ringer's), and eye ointment prior to surgery. Body temperature was maintained throughout the procedure using a feedback-controlled heating pad. After anesthetizing with 2% isofluorane, ear bars were fitted to temporarily stabilize the head axis. First, local topical and injectable lidocaine was administered, and then the intact skull was surgically exposed and reinforced with Vetbond (3 M, Vetbond, 1469SB). V1-adjacent regions were additionally reinforced with a layer of dental acrylic (Lang Ortho-Jet Powder and Ortho-Jet Powder Liquid). Headplates were then affixed with the 4-5mm window centered over V1 using dental acrylic. Lastly, V1 windows were sealed with a final layer of Vetbond, and mice returned to heated home cages upon waking. For consistency, all headplates were installed on the right hemisphere only. Mice received subcutaneous injections of Carprofen to reduce pain and inflammation (up to 3 days) after installation.

Intrinsic Signal Optical Imaging (ISOI) for Intracerebral Procedures: To precisely target V1, the central region (bV1) was physiologically determined for each individual mouse using ISOI. After inducing and maintaining a steady plane of light anesthesia with 0.8-1.2% isofluorane, headplated mice were secured into the imaging rig. Headplate windows were filled with PBS and covered with a 10 mm glass coverslip. PBS was added as needed throughout sessions to keep coverslios level with the top of the headplate. Eye moisture was maintained using periodic silicone oil application, and body temperature was regulated by a feedback-controlled heating pad.

Intrinsic signal images were collected using a custom-designed macroscope (Nikon 135×50 mm lenses) equipped with a Dalsa 1M 30 CCD camera positioned over the headplate. First, a green (530 nm) light-emitting diode (LED) was used to visualize and capture an image of surface vasculature. Then, the camera was refocused ~450-550 um below the pia surface to target layer II/III of bV1, and a red (617 nm) LED light was used to acquire the intrinsic signal. Visual stimuli were displayed on an Acer V193 monitor (53×33 cm, 60 Hz refresh rate, 20 cd/m2 mean luminance) positioned 25 cm in front of the mice, aligned to their midline and eye line. Stimuli were generated using MATLAB Psychophysics Toolbox extensions and consisted of contrast-modulating sweeping noise restricted to −5° to +15° visual field azimuth and −18° to +36° visual field elevation. Each imaging session was a 5 minute presentation of stimuli to both eyes. Custom-written Matlab scripts using Fourier analysis were used to generate phase maps of bV1 responses. Resulting phase maps were then overlaid on top of vasculature images to visualize bV1 bounds and determine optimal injection sites.

Inhibitory neuron transplantation for transcriptomics: to generate donor embryonic tissue, VGAT-cre females were briefly cohoused with cre-dependent tdTomato males overnight (<20 hrs). Upon successful breeding, pregnant dams were sacrificed ~2 wks later by isofluorane and cervical dislocation. E13.5-14.5 VGAT-tdTomato embryos were quickly dissected out into chilled L-15 solution (Gibco, 21083027), and forebrains microdissected into a second petri dish of chilled L-15. tdTomato signal was confirmed in each forebrain prior to MGE and LGE microdissection using an epifluorescence scope. Isolated MGE and LGE tissue were then collected in separate tubes of L-15 with 2% (v/v) of DNase I (Roche, 0471672800) and placed on ice.

Headplated adult VGAT-ZsGreen mice were anesthetized with 2% isoflurane and secured into the surgical rig. All recipients received pre-operative Carprofen, Ringer's, and eye ointment. Body temperature was maintained throughout the procedure using a feedback-controlled heating pad, and anesthesia was gradually reduced as needed. To allow the injection micropipette to penetrate, skull underlying mapped bV1 injection sites were thinned using a dental drill (Midwest, 78044) and FG1/4 carbide burr.

Glass micropipettes (Wiretrol 5 μl, Drummond Scientific Company) were pulled with ~75 um diameter tips and beveled. To dissociate donor tissue into injectable cell suspensions, isolated MGE or LGE were front-loaded into prepared micropipettes immediately prior to transplantation. Loaded micropipettes were then angled perpendicularly, relative to the brain surface curvature of each individual recipient. Injections into mapped bV1 sites were performed using a custom-made hydraulic manipulator (Narishige, MO-10) between ~400-600 um below the surface at 10 nl/min. Each recipient received 100 nl per injection for a total of 2 injections (~2 isolated MGEs/LGEs). After injection, micropipettes were incrementally withdrawn to prevent backflow, and sites were resealed with Vetbond. Post-operative mice were returned to heated home cages upon waking, and received subcutaneous injections of Carprofen to reduce pain and inflammation (up to 3 days) after installation.

To maintain consistency, experimental agents (MGE, LGE, saline) were injected only into V1 on the right hemisphere. Each transplant cohort included a balanced number of MGE, LGE, and non-transplanted recipients.

V1 isolation, Dissociation. Cell Sorting, and RNA Extraction: at Their Designated experimental timepoints, mice (35 DAT, 70 DAT, non-transplanted littermates) were sacrificed via 1.2% tribromoethanol. After injection, mice were transcardially perfused with RNAase inhibitor and transcriptional inhibitors diluted in chilled L-15 solution for 5 min. To mitigate cell cycle discrepancies, perfusions for each collection cohort began at the same time of day and were completed within 2 hrs. Brains were then dissected out, and Vis isolated using stereotaxic coordinates centered on mapped bV1 (3-4mm lateral, −0.5-0.5mm anterior to posterior bregma). Isolated V1 tissue was then homogenized into smaller pieces and suspended in an additional L-15/inhibitors solution in gentleMACS C tubes (Miltenyi Biotec). Tissue chunks were further dissociated using the gentleMACS Octo Dissociator with Heaters (Miltenyi Biotec) and Adult Brain Dissociation Kit (mouse and rat, Miltenyi Biotec). Resulting cell suspensions were incubated with microglial antibody (Cd11b-APC) and cell viability indicator DAPI for 15 min.

Immunolabeled samples were then further refined via fluorescence. Following standard FACS screening for cell viability/doublets and fluorescence-gating based on control samples, samples were separated by cell population (ZsGreen-host inhibitory neurons, APC-microglia). All sorted populations were collected into 1 ml RNA stabilizer Trizol on ice. Finally, RNA was extracted from each sample using the RNA Clean & Concentrator kit and promptly stored at −80C.

Immunohistochemistry: Mice were sacrificed via 1.2% tribromoethanol (Avertin, 18 ml/g). After injection, mice were transcardially perfused with chilled 4% PFA in PBS for 5 minutes at 6ml/min. Collected brains were post-fixed overnight and cryoprotected with 30% sucrose. Brains were sectioned coronally into 30 um free-floating sections using a freezing sliding microtome (Microm, HM450). To reduce batch effects, samples across experimental/age groups were sorted into mixed batches using a custom-written randomization script. To representatively sample V1, sections were selected every ~200 um from anterior to posterior V1. V1 sections were then permeabilized with 0.3% Triton-X in PBS for 1 hr, and blocked with 1% normal donkey serum in 0.3% Triton-X/PBS. Following overnight incubation with primary antibodies at 4 C, sections were washed 3× in 0.3% Triton-X/PBS and incubated with secondary antibodies for 2 hr at room temperature. Sections were again washed 3× in 0.3% Triton-X/PBS, then mounted and coverslipped using Fluoroshield with DAPI (Millipore-Sigma, F6057).

To visualize Calb1 protein expression, sections were incubated with donkey anti-rabbit Calb1 primary antibody, followed by donkey anti-rabbit Alexa 647 secondary antibody. To visualize transplanted cells, sections were incubated with donkey anti-RFP (Fisher MA5-15257, 1:250) to amplify the VGAT-tdTomato signal, followed by donkey anti-mouse 568 secondary antibody. Given the intensity of VGAT-ZsGreen signal, host cells were visualized without additional amplification.

Confocal microscopy and image analysis: stained sections were imaged using a Leica TCS SP8 confocal microscope (20× immersion objective). LAS X image processing software (Leica Microsystem) was used to perform image tiling and acquire z-stacks (20-30 um) of V1 and adjacent landmarks. To remain blind to the experimental group (i.e. transplant vs. non-transplanted), Calbi channels were separated out from exported images and quantified together. Stacks were first z-projected (maximum intensity), then V1 boundaries and cortical layers annotated (Allen Brain Atlas reference). Next, a minimal Gaussian blur (1 unit) was applied to smooth background noise. Cells with signal intensity above background were counted as Calb1+ (cell counter plugin, Fiji).

To determine colocalization, tdTomato or ZsGreen cells were assessed for Calb1+ signal and counted. The percentage of transplanted tdTomato cells that expressed Calb1 was calculated by dividing the number of Calb1+/tdTomato+ cells by the total number of tdTomato+ cells. The percentage of host ZsGreen cells that expressed Calb1 was calculated by dividing the number of Calb1+/ZsGreen+ cells by the total number of ZsGreen+ cells.

Intracerebral Viral Injection: To upregulate Calb1 expression specifically within adult inhibitory neurons, P120-180 non-transplanted VGAT-tdTomato mice were injected with a cre-restricted, Calb1-overexpressing virus (AAV1-CMV-DIO-mCalb1-T2A-eGFP). An eGFP fluorescent tag was included in the polycistronic construct to visualize successful AAV incorporation and expression. After headplate installation and V1 mapping, adult VGAT-tdTomato mice were anesthetized with 2% isoflurane and secured into the surgical rig. All recipients received pre-operative Carprofen, Ringer's, and eye ointment. Body temperature was maintained throughout the procedure using a feedback-controlled heating pad, and anesthesia was gradually reduced as needed. To allow the injection micropipette to penetrate, skull underlying mapped bV1 injection sites were thinned using a dental drill (Midwest, 78044) and FG1/4 carbide burr.

Glass micropipettes (Wiretrol 5 μl, Drummond Scientific Company) were pulled with ~75 um diameter tips and beveled. Virus was front-loaded into prepared micropipettes and angled perpendicularly, relative to the brain surface curvature of each individual recipient. Injections into mapped bV1 sites were performed using a custom-made hydraulic manipulator (Narishige, MO-10) between ~400-600 um below the surface at 10 nl/min. Each recipient received 200nl per injection for a total of 2 injections. To maintain consistency, experimental agents (AAV, saline) were injected only into V1 on the right hemisphere. After injection, micropipettes were incrementally withdrawn to prevent backflow, and sites were resealed with Vetbond. Post-operative mice were returned to heated home cages upon waking, and received subcutaneous injections of Carprofen to reduce pain and inflammation (up to 3 days) after installation. Mice were returned to normal routine for ~3wks while viral expression stabilized.

Intrinsic signal optical imaging (soi) for ocular dominance plasticity: to assess whether experimental mice exhibit OD plasticity, ISOI was performed before and after monocular deprivation (MD) to measure any changes in OD indices. After inducing and maintaining a steady plane of light anesthesia with 0.8-1.2% isofluorane, headplated mice were secured into the imaging rig. Headplate windows were filled with PBS and covered with a 10 mm glass coverslip. PBS was added as needed throughout sessions to keep coverslips level with the top of the headplate. Eye moisture was maintained using periodic silicone oil application, and body temperature was regulated by a feedback-controlled heating pad.

Intrinsic signal images were collected using a custom-designed macroscope (Nikon 135*50 mm lenses) equipped with a Dalsa 1M30 CCD camera positioned over the headplate. First, a green (530 nm) light-emitting diode (LED) was used to visualize and capture an image of surface vasculature. Then, the camera was refocused ~450-550 μm below the pia surface to target layer II/III of bV1, and a red (617 nm) LED light was used to acquire the intrinsic signal. Visual stimuli were displayed on an Acer V193 monitor (53×33 cm, 60 Hz refresh rate, 20 cd/m2 mean luminance) positioned 25 cm in front of the mice, aligned to their midline and eye line. To capture eye-specific responses, a single eye block was positioned in front of one eye or the other (alternating) during stimuli presentation and recording.

Stimuli was generated using MATLAB Psychophysics Toolbox extensions and consisted of contrast-modulating sweeping noise restricted to−5° to +15° visual field azimuth and −18° to +36° visual field elevation. Each recording trial was a 5 minute presentation of stimuli at 0° and 180°, with a total of 3-4 recordings for each eye. Custom-written Matlab scripts using Fourier analysis were used to generate amplitude and phase maps of bV1 responses. Fourier maps were then smoothed with 5×5 Gaussian kernels to compute final amplitude maps. To account for hemodynamic delay, phase maps were normalized by subtracting the phase of cortical responses at 180° from the phase at 0°. Maps of the absolute retinotopic phase were shown in terms of visual angle relative to the center of the monitor.

The ODI for each animal was calculated as (C−1)/(C+D), where C is the average response amplitude for the contralateral eye and I is the average response amplitude for the ipsilateral eye, each across 3-4 trials. OD shift was calculated by subtracting PreMD ODI from PostMD ODI.

Monocular Deprivation: to reveal the presence or absence of underlying circuit plasticity, experimental mice were subjected to a temporary change in visual experience. Immediately following preMD imaging, the eye contralateral to the injected hemisphere was sutured using Perma-Hand Silk (Ethicon, K809H). While under anesthesia, mice were quickly transferred from the imaging rig to the surgical rig. 2% isoflurane, pre-operative Carprofen, and Ringer's were administered. Body temperature was maintained throughout the procedure using a feedback-controlled heating pad. Two mattress sutures were applied across the contralateral eyelid (one on each side) and the final knot was additionally secured with a dab of Vetbond. Post-operative mice were returned to heated home cages upon waking, and received subcutaneous injections of Carprofen to reduce pain and inflammation (up to 3 days) if needed.

Eyelid sutures were monitored daily for integrity, and carefully removed after 3 days. To allow the eye to fully open before postMD imaging, mice were returned to their heated home cage for 30 minutes after suture removal. Mice with prematurely-opened sutures, cataracts, cloudy eyes, or drooping eyelids were excluded from postMD imaging.

Statistics: Experiments were replicated successfully with multiple animals. All quantification and data analyses were performed in a blind and unbiased manner. To test for the significant differences between the two experimental groups, either a normal t-test with Welch's correction (normally distributed) or Mann-Whitney rank test (not normally distributed) was used. To test for the significant differences between three or more groups, either a normal one-way ANOVA followed by a Holm-Sidak's multiple comparisons test (normally distributed) or Kruskal-Wallis one-way ANOVA corrected using Dunn's multiple comparisons test (not normally distributed) was used.

Isolation of host inhibitory neurons from transplanted adult V1: To genetically label host Inhibitory neurons in designated transplant recipients, transgenic mice expressing Cre-recombinase under control of the vesicular GABA transporter (VGAT) promoter were crossed with mice carrying Cre-dependent ZsGreen (FIGS. 1A and 1B). Upon adulthood (~p120), resulting VGAT-ZsGreen littermates were affixed with custom-printed headplates centered over V1 on the right hemisphere (FIGS. 1A and 1B). To account for anatomical variation between mice, V1 boundaries were physiologically determined prior to transplantation via intrinsic signal optical imaging (ISOI) (FIGS. 1A and 1B). This functional imaging method leverages hemodynamic changes as a readout for neural activity and can be combined with visual stimuli to assess V1 responses. Generated retinotopic maps of binocular V1 were then superimposed onto images of surface vasculature to determine optimal injection sites.

Donor tissue was microdissected from timed E13.5-14.5 embryos (FIGS. 1A and 1B). Dissociated donor tissue was then transplanted into adult VGAT-ZsGreen recipients who had received headplates at least a week prior. Each recipient cohort included: 1) non-transplanted, age-matched controls to account for age-related gene expression and 2) LGE transplanted controls to account for cell transplantation-related gene expression.

In studies that use mouse V1 as a model, visual deprivation is often used to reveal the underlying plasticity of local circuitry. However, the mice were not subjected to such perturbations to avoid confounding gene expression changes that establish a plastic state, with changes that result from triggering that plasticity. After returning to normal routine post-transplantation, recipient cohorts were later euthanized and perfused at timepoints that represent two distinct physiological states-either: a) 35 days after transplantation, during the peak of the transplant-reactivated critical period (35 DAT), or b) 70 days after transplantation, once the reactivated period had closed (70 DAT) (Fig). To preserve the transcriptomic integrity of these in vivo states, all mice were perfused immediately with transcription and RNAase inhibitors, and without fixation.

V1s were collected solely from the right hemisphere, even in non-transplanted mice, to preclude variation from hemispheric asymmetry. Due to the small size of V1 tissue post-dissection (~1 cm³), Vis were pooled from the same experimental groups together to ensure quality downstream processing. As such, each n reported represents 2-3 biological replicates each. V1 samples were then dissociated and sorted into separate cell populations via fluorescence-activated cell sorting (FACS). Finally, high-quality RNA samples were isolated (RIN>8) from collected cells and prepared into bulk sequencing libraries.

Calbindin 1 (Calb1): Calb1, is known for its potent Ca²⁺-binding properties, is a mobile, cytosolic protein that localizes to the soma, dendrites, and spines of predominantly GABAergic neurons. In comparison with other prominent EF-hand family members, PV, and calretinin, Calb1 has been shown to selectively buffer fast synaptically-evoked Ca²⁺ transients. Loss of Calb1 impairs multiple aspects of synaptic plasticity, such as proper LTP maintenance, paired-pulse facilitation, and neuronal excitability—which ultimately manifest as functional deficits in learning and memory, motor coordination, and sensory integration.

Within the central nervous system, Calb1 expression is broadly reported to increase during embryogenesis, peak amidst postnatal development, and sharply downregulate upon adulthood. This downregulation is distinct from other Ca²⁺-binding proteins, such as PV, that do not decrease with age. Importantly, Calb1's reduction across brain regions has been attributed to programmed downregulation rather than neuronal loss. As such, Calb1 appeared uniquely positioned at the intersection of both GO predictions and DE comparisons for initial candidate investigation

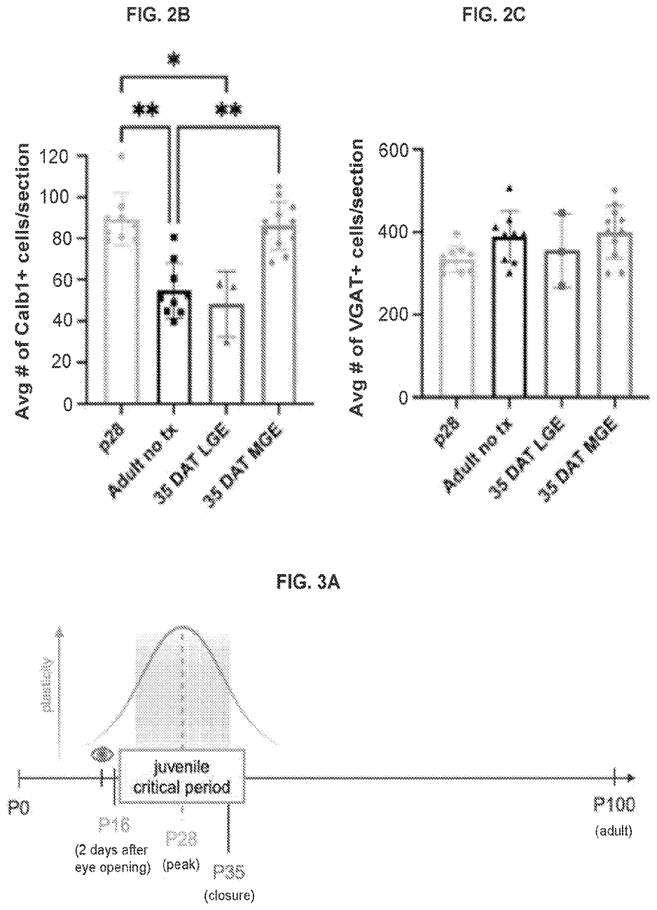

Immunostaining confirms Calb1 upregulation in MGE transplant recipients: To independently validate the RNAseq results, Calb1 was assessed by immunostaining V1 sections from adult MGE, LGE, and non-transplanted mice 35 DAT. In MGE transplant recipients, levels of Calb1 expression are significantly higher than both LGE and non-transplanted controls (FIG. 2A). LGE transplantation does not significantly alter Calb1 expression levels from the non-transplanted baseline (FIG. 2B). Quantification of VGAT+ cells (transplant and host) across groups confirms that this increase in Calb1 expression following transplantation is not due to significantly different numbers of inhibitory neurons (FIG. 2C). Calb1 immunostaining in non-transplanted controls shows a distinct laminar distribution with enrichment in cortical layers IV/III and V, wherein MGE transplantation appears to significantly increase Calb1 expression in deeper layers (FIG. 2A). Calb1 immunostaining across groups is restricted to inhibitory neurons and segregates into strongly and weakly expressing Calb1 cells. Strongly-expressing Calb1 cells appear primarily localized to deeper layers while weakly-expressing Calb1 cells constitute the majority of the superficial population (FIG. 2A).

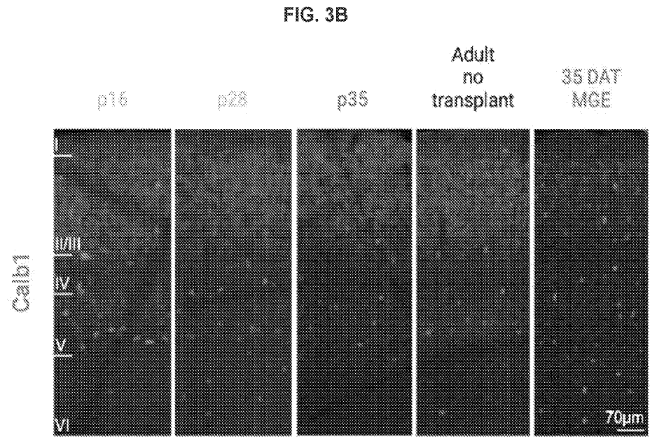

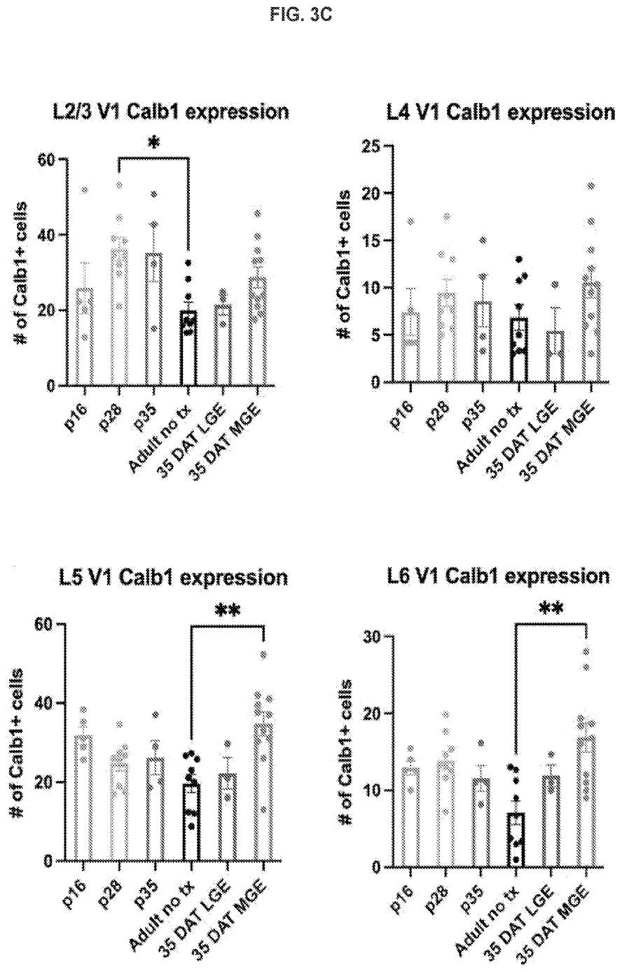

Postnatal V1 Calb1 expression peaks during the critical period: While Calb1 function has been relatively more explored in the hippocampus and cerebellum, little work (beyond application as a neuroanatomical marker) has been done in the mouse visual cortex. MGE-upregulated Calb1 levels were compared to levels during key postnatal timepoints for visual development: p16 (two days after eye-opening, onset of visual experience), p28 (peak of the juvenile critical period), and p35 (closure of the juvenile critical period) (FIG. 3A). Interestingly, Calb1 expression has an appreciable peak at p28 and is downregulated already by p35 (FIG. 3B). p35 Calb1 levels do not appear significantly different from p100 Calb1 levels. Reduced Calb1 upon adulthood appears to be mostly mediated by a loss of expression in deeper cortical layers (FIG. 3C). Notably, MGE-upregulated Calb1 levels are not significantly different from p28 expression (FIG. 2B), suggesting that MGE transplantation restores adult host Calb1 expression to juvenile levels.

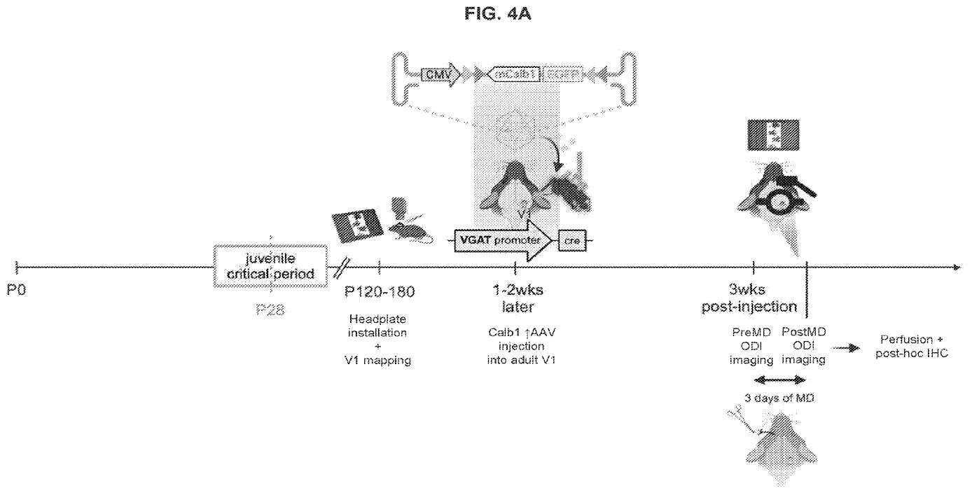

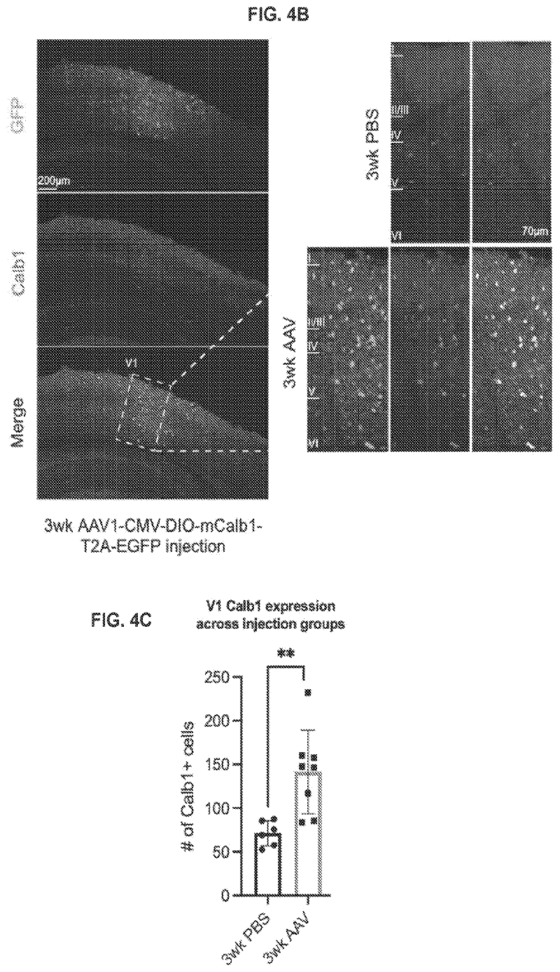

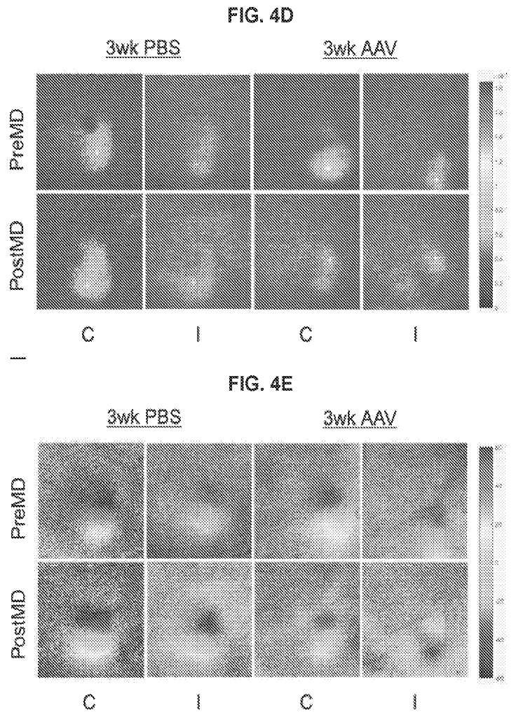

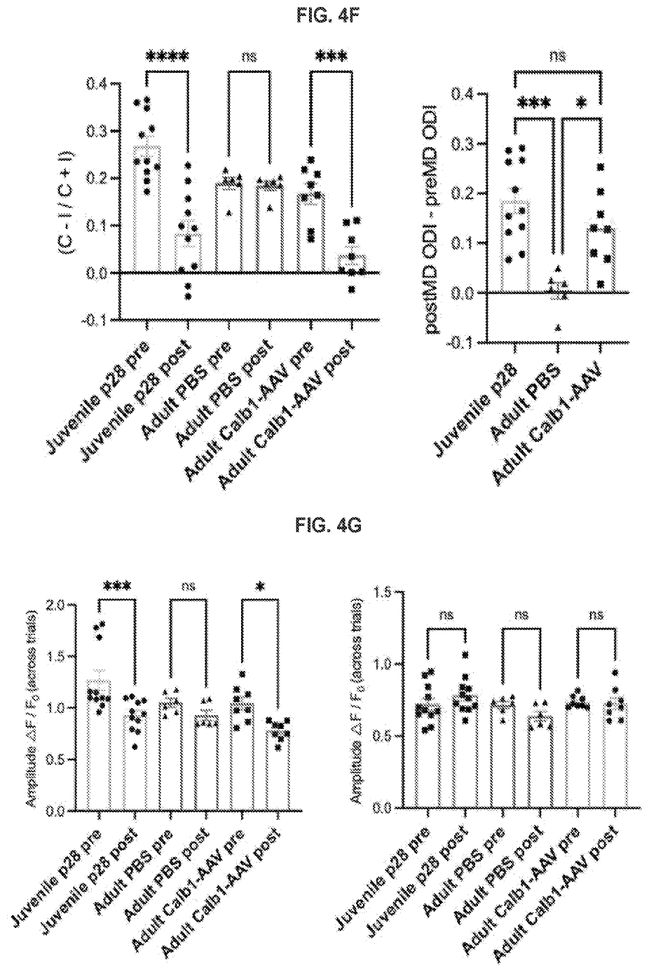

Upregulation of Calb1 in adult V1 inhibitory neurons restores critical period plasticity: To restrict Calb1 overexpression to inhibitory neurons, binocular V1 was mapped out in non-transplanted, adult VGAT-cre mice and injected cre-dependent Calbβ1-overexpressing AAV (AAV1-CMV-DIO-mCalb1-T2A-eGFP) (FIG. 4A). Successful upregulation of Calb1 and spread of the GFP reporter were established during initial viral optimization and confirmed after each experiment via post-hoc immunohistochemistry FIG. 4B). 3 weeks after injection, baseline V1 responses were recorded from each eye using ISOI. Contralateral and ipsilateral responses were used to calculate an ocular dominance index (ODI), a measure of eye-specific response strength relative to each other. Immediately following baseline imaging, the eye contralateral to the injected hemisphere was sutured shut and briefly deprived of visual stimuli. 3 days of monocular deprivation (MD) has been previously shown to distinguish juvenile OD plasticity from other adult-plasticity mechanisms.

After 3 days of MD, eyelid sutures were removed, and V1 responses were recorded from each eye again. postMD ODIs were compared to preMD ODIs to determine whether those mice had undergone any shifts in OD. Saline-injected adult mice do not exhibit OD plasticity following MD (FIG. 4F). Surprisingly, AAV-injected adult mice show a significant shift in OD towards the non-deprived eye, indicating robust reactivation of critical period plasticity (FIG. 4F). Closer analysis of eye-specific response strengths pre and postMD suggests that the OD shift following Calb1 upregulation is primarily mediated by a decrease in response from the deprived eye (FIG. 4G). While OD shifts in adult mice require longer deprivation and involve a gain in response from the non-deprived eye, loss of response from the deprived eye is more characteristic of OD shifts occurring within the juvenile critical period.

### EXAMPLE 2

The following is a non-limiting example of the present invention. It is to be understood that said example is not intended to limit the present invention in any way. Equivalents or substitutes are within the scope of the present invention.

A group of patients with mild to moderate Alzheimer's disease are administered a series of gene therapy agents aimed at enhancing Calb1 expression. The gene therapy agent is administered weekly over a period of 4 weeks. Following the treatment regimen, functional MRI scans and cognitive function of the patients are assessed with neuropsychological tests, with both the patient and administering physicians blinded to whether the patient received the therapy or a placebo. After treatment, the obtained measurements are compared with baseline data collected before the initiation of treatment. This comparison reveals significant improvements in daily functions among patients who received the gene therapy agent. No side effects reported.

### EXAMPLE 3

The following is a non-limiting example of the present invention. It is to be understood that said example is not intended to limit the present invention in any way. Equivalents or substitutes are within the scope of the present invention.

A 28-year-old male diagnosed with epilepsy receives a prescription for a novel composition comprising a small molecule aimed at elevating the levels of Calbindin in a subset of his neuronal cells. The composition comprising a small molecule is administered once daily. Following two months of treatment, the patient returns for a follow-up appointment and reports a reduction in overall symptoms. The doctor orders confirmatory tests to validate this anecdotal improvement. Upon reviewing the test results, the doctor confirms the reduction in symptoms and notes favorable brain scan findings. No side effects reported.

### EXAMPLE 4

The following is a non-limiting example of the present invention. It is to be understood that said example is not intended to limit the present invention in any way. Equivalents or substitutes are within the scope of the present invention.

A cohort of patients diagnosed with mild to moderate Parkinson's disease undergo a series of gene therapy administrations aimed at upregulating the expression of one or a combination of genes selected from a set including Calb1, Grin2b, Itpka, Neurod6, Panx2, Msi1, Cck, Nrgn, Caln1, Thy1, Marcks11, Ncs1, Ctbp2, Git2, Nfkb1, Tgfbr1, Tgfbr2, Tgfb1, and Dig4. These gene therapy agents are administered weekly over an 8-week period. Subsequently, functional MRI scans and neuropsychological tests are conducted to assess cognitive function, with both the patients and administering physicians blinded to the treatment allocation. Post-treatment, the obtained measurements are compared with baseline data collected before the treatment initiation, revealing significant improvements in daily functions among patients who received the gene therapy agent. No side effects reported.

### Embodiments

The following embodiments are intended to be illustrative only and not to be limiting in any way.

Embodiment 1: A method of increasing neural plasticity in a subject in need thereof, the method comprising: administering a gene therapy agent to the subject, said gene therapy agent configured to modulate (e.g., increase) the expression of calbindin in a population of neuronal cells in the subject, wherein modulating (e.g., increasing) the expression of calbindin increases neural plasticity in the subject.

Embodiment 2: A method of increasing neural plasticity in a subject in need thereof, the method comprising: administering a gene therapy agent to the subject, said gene therapy agent configured to modulate (e.g., increase) the expression of one or a combination of genes selected from a group consisting of Calb1, Grin2b, Itpka, Neurod6, Panx2, Msi1, Cck, Nrgn, Caln1, Thy1, Marcksi1, Ncs1, Ctbp2, Git2, Nfkb1, Tgfbr1, Tgfbr2, Tgfb1, and Dig4 in a population of neuronal cells in the subject, wherein modulating (e.g., increasing) the expression of one or a combination of genes selected from a group consisting of Calb1, Grin2b, Itpka, Neurod6, Panx2, Msi1, Cck, Nrgn, Cain1, Thy1, Marcks11, Ncs1, Ctbp2, Git2, Nfkb1, Tgfbr1, Tgfbr2, Tgfb1, and Dig4 increases neural plasticity in the subject.

Embodiment 3: A method of increasing neural plasticity in a subject in need thereof, the method comprising: administering a gene therapy agent to the subject, said gene therapy agent configured to modulate (e.g., increase) the expression of one or a combination of genes selected from a group consisting of Grin2b, Itpka, Neurod6, Panx2, Msi1, Cck, Nrgn, Cain1, Thy1, Marcksi1, Nos1, Ctbp2, Git2, Nfkb1, Tgfbr1, Tgfbr2, Tgfb1, and Dig4 in a population of neuronal cells in the subject, wherein modulating (e.g., increasing) the expression of one or a combination of genes selected from a group consisting of Grin2b, Itpka, Neurod6, Panx2, Msi1, Cck, Nrgn, Caln1, Thy1, Marcksi1, Ncs1, Ctbp2, Git2, Nfkb1, Tgfbr1, Tgfbr2, Tgfb1, and Dig4 increases neural plasticity in the subject.

Embodiment 4: The method of any one of embodiments 1-3, wherein the modulating (e.g., increasing) the expression of calbindin or one or a combination of genes selected from a group consisting of Grin2b, Itpka, Neurod6, Panx2, Msi1, Cck, Nrgn, Caln1, Thy1, Marcksi1, Ncs1, Ctbp2, Git2, Nfkb1, Tgfbr1, Tgfbr2, Tgfb1, and Dig4 increases neural plasticity in the population of neuronal cells in the subject.

Embodiment 5: A method of increasing neural plasticity in a subject in need thereof, the method comprising: administering a gene therapy agent to the subject, said gene therapy agent configured to modulate (e.g., increase) the expression of calbindin in a population of neuronal cells in the subject, wherein modulating (e.g., increasing) the expression of calbindin increases neuronal plasticity in the subject.

Embodiment 6: A method of increasing neural plasticity in a subject in need thereof, the method comprising: administering a gene therapy agent to the subject, said gene therapy agent configured to modulate (e.g., increase) the expression of one or a combination of genes selected from a group consisting of Calb1, Grin2b, Itpka, Neurod6, Panx2, Msi1, Cck, Nrgn, Caln1, Thy1, Marcksl1, Ncs1, Ctbp2, Git2, Nfkb1, Tgfbr1, Tgfbr2, Tgfb1, and Dig4 in a population of neuronal cells in the subject, wherein modulating (e.g., increasing) the expression of one or a combination of genes selected from a group consisting of Calb1, Grin2b, Itpka, Neurod6, Panx2, Msi1, Cck, Nrgn, Caln1, Thy1, Marcksi1, Ncs1, Ctbp2, Git2, Nfkb1, Tgfbr1, Tgfbr2, Tgfb1, and Dig4 increases neuronal plasticity in the subject.

Embodiment 7: A method of increasing neural plasticity in a subject in need thereof, the method comprising: administering a gene therapy agent to the subject, said gene therapy agent configured to modulate (e.g., increase) the expression of one or a combination of genes selected from a group consisting of Grin2b, Itpka, Neurod6, Panx2, Msi1, Cck, Nrgn, Caln1, Thy1, Marcksl1, Ncs1, Ctbp2, Git2, Nfkb1, Tgfbr1, Tgfbr2, Tgfb1, and Dig4 in a population of neuronal cells in the subject, wherein modulating (e.g., increasing) the expression of one or a combination of genes selected from a group consisting of Grin2b, Itpka, Neurod6, Panx2, Msi1, Cck, Nrgn, Caln1, Thy1, Marcksl1, Ncs1, Ctbp2, Git2, Nfkb1, Tgfbr1, Tgfbr2, Tgfb1, and Dig4 increases neuronal plasticity in the subject.

Embodiment 8: The method of any one of embodiments 1-7, wherein the modulating (e.g., increasing) the expression of calbindin or one or a combination of genes selected from a group consisting of Grin2b, Itpka, Neurod6, Panx2, Msi1, Cck, Nrgn, Cain1, Thy1, Marcksl1, Ncs1, Ctbp2, Git2, Nfkb1, Tgfbr1, Tgfbr2, Tgfb1, and Dig4 modulates (increases) experience-dependent plasticity in the population of neuronal cells in the subject Embodiment 9: A method of increasing neural plasticity in a subject in need thereof, the method comprising: administering a gene therapy agent to the subject, said gene therapy agent configured to modulates (e.g., increases) the expression of calbindin in a population of neuronal cells in the subject, wherein modulating (e.g., increasing) the expression of calbindin increases experience-dependent plasticity in the subject.

Embodiment 10: The method of any one of embodiments 1-9, wherein the gene therapy agent modulates (e.g., increases) the expression of one or a combination of genes selected from a group consisting of Grin2b, Itpka, Neurod6, Panx2, Msi1, Cck, Nrgn, Caln1, Thy1, Marcksl1, Ncs1, Ctbp2, Git2, Nfkb1, Tgfbr1, Tgfbr2, Tgfb1, and Dig4.

Embodiment 11: A method of increasing neural plasticity in a subject in need thereof, the method comprising: administering a gene therapy agent to the subject, said gene therapy agent configured to modulate (e.g., increase) the expression of one or a combination of genes selected from a group consisting of Calb1, Grin2b, Itpka, Neurod6, Panx2, Msi1, Cck, Nrgn, Caln1, Thy1, Marcksi1, Ncs1, Ctbp2, Git2, Nfkb1, Tgfbr1, Tgfbr2, Tgfb1, and Dig4, wherein modulating (e.g., increasing) the expression of of one or a combination of genes selected from a group consisting of Calb1, Grin2b, Itpka, Neurod6, Panx2, Msi1, Cck, Nrgn, Caln1, Thy1, Marcksl1, Ncs1, Ctbp2, Git2, Nfkb1, Tgfbr1, Tgfbr2, Tgfb1, and Dig4 increases experience-dependent plasticity in the subject.

Embodiment 12: The method on any one of embodiments 1-11, wherein the population of neuronal cells comprises excitatory neurons, inhibitory neurons, or a combination thereof.

Embodiment 13: The method on any one of embodiments 1-12, wherein the therapy agent comprises viral vectors, exosomes, nanoparticles, small interfering RNAs (siRNAs) and microRNAs (miRNAs), antisense oligonucleotides (ASOs), aptamers, modified cells, or the like.

Embodiment 14: The method of any one of embodiments 1-13, wherein the gene therapy agent directly modulates (increases) the expression of calbindin or one or a combination of genes selected from a group consisting of Grin2b, Itpka, Neurod6, Panx2, Msi1, Cck, Nrgn, Caln1, Thy1, Marcksl1, Ncs1, Ctbp2, Git2, Nfkb1, Tgfbr1, Tgfbr2, Tgfb1, and Dig4.

Embodiment 15: The method of any one of embodiments 1-13, wherein the gene therapy agent indirectly modulates (increases) the expression of calbindin or one or a combination of genes selected from a group consisting of Grin2b, Itpka, Neurod6, Panx2, Msi1, Cck, Nrgn, Caln1, Thy1, Marcksl1, Ncs1, Ctbp2, Git2, Nfkb1, Tgfbr1, Tgfbr2, Tgfb1, and Dig4.

Embodiment 16: A method of increasing neural plasticity in a subject in need thereof, the method comprising: administering a viral vector to the subject, said viral vector configured to modulate (increase) the expression of calbindin (Calb1) in a population of neuronal cells in the subject, wherein modulating (increasing) the expression of Calb1 increases a level of experience-dependent plasticity in the subject.

Embodiment 17: The method of embodiment 16, wherein the viral vector directly increases the expression of calbindin.

Embodiment 18: The method of embodiment 16, wherein the viral vector indirectly increases the expression of calbindin.

Embodiment 19: The method of any one of embodiments 16-18, wherein the viral vector further modulates the expression of one or a combination of genes selected from a group consisting of Grin2b, Itpka, Neurod6, Panx2, Msi1, Cck, Nrgn, Cain1, Thy1, Marcksi1, Ncs1, Ctbp2, Git2, Nfkb1, Tgfbr1, Tgfbr2, Tgfb1, and Dig4.

Embodiment 20: A method of increasing neural plasticity in a subject in need thereof, the method comprising: administering a viral vector to the subject, said viral vector configured to modulate (increase) the expression of one or a combination of genes selected from a group consisting of Calb1, Grin2b, Itpka, Neurod6, Panx2, Msi1, Cck, Nrgn, Caln1, Thy1, Marcksl1, Ncs1, Ctbp2, Git2, Nfkb1, Tgfbr1, Tgfbr2, Tgfb1, and Dig4 in a population of neuronal cells in the subject, wherein modulating (increasing) the expression of one or a combination of genes selected from a group consisting of Calb1, Grin2b, Itpka, Neurod6, Panx2, Msi1, Cck, Nrgn, Caln1, Thy1, Marcksi1, Nos1, Ctbp2, Git2, Nfkb1, Tgfbr1, Tgfbr2, Tgfb1, and Dig4 increases a level of experience-dependent plasticity in the subject.

Embodiment 21: The method of embodiment 20, wherein the viral vector directly modulates (increases) the expression of one or a combination of genes selected from a group consisting of Calb1, Grin2b, Itpka, Neurod6, Panx2, Msi1, Cck, Nrgn, Caln1, Thy1, Marcksi1, Ncs1, Ctbp2, Git2. Nfkb1, Tgfbr1, Tgfbr2, Tgfb1, and Dig4.

Embodiment 22: The method of embodiment 20, wherein the viral vector indirectly modulates (increases) the expression of one or a combination of genes selected from a group consisting of Calb1, Grin2b, Itpka, Neurod6, Panx2, Msi1, Cck, Nrgn, Caln1, Thy1, Marcksl1, Ncs1, Ctbp2, Git2, Nfkb1, Tgfbr1, Tgfbr2, Tgfb1, and Dig4.

Embodiment 23: The method on any one of embodiments 16-22, wherein the population of neuronal cells comprises excitatory neurons, inhibitory neurons, or a combination thereof.

Embodiment 24: The method of any one of embodiments 16-23 further comprising measuring the expression of one or a combination of genes selected from a group consisting of Calb1, Grin2b, Itpka, Neurod6, Panx2, Msi1, Cck, Nrgn, Cain1, Thy1, Marcksl1, Ncs1, Ctbp2, Git2, Nfkb1, Tgfbr1, Tgfbr2, Tgfb1, and Dig4 in the population of neuronal cells prior to the administration of the viral vector.

Embodiment 25: A method of increasing neural plasticity in a subject in need thereof, the method comprising: administering exosomes to the subject, said exosomes configured to modulate (increase) the expression of calbindin (Calb1) in a population of neuronal cells in the subject, wherein modulating (increasing) the expression of Calb1 increases a level of experience-dependent Plasticity in the Subject.

Embodiment 26: The method of embodiment 25, wherein the exosomes directly modulate (e.g., increase) the expression of Calb1.

Embodiment 27: The method of embodiment 25, wherein the exosomes indirectly modulate (e.g., increase) the expression of Calb1.

Embodiment 28: The method of any one of embodiments 25-27, wherein the exosomes further modulate (e.g., increase) the expression of one or a combination of genes selected from a group consisting of Grin2b, Itpka, Neurod6, Panx2, Msi1, Cck, Nrgn, Cain1, Thy1, Marcksl1, Ncs1, Ctbp2, Git2, Nfkb1, Tgfbr1, Tgfbr2, Tgfb1, and Dig4.

Embodiment 29: A method of increasing neural plasticity in a subject in need thereof, the method comprising: administering exosomes to the subject, said exosomes configured to modulate (increase) the expression one or a combination of genes selected from a group consisting of Calb1, Grin2b, Itpka, Neurod6, Panx2, Msi1, Cck, Nrgn, Caln1, Thy1, Marcksl1, Ncs1, Ctbp2, Git2, Nfkb1, Tgfbr1, Tgfbr2, Tgfb1, and Dig4 in a population of neuronal cells in the subject, wherein modulating (increasing) the expression of one or a combination of genes selected from a group consisting of Calb1, Grin2b, Itpka, Neurod6, Panx2, Msi1, Cck, Nrgn, Caln1, Thy1, Marcksi1, Ncs1, Ctbp2, Git2, Nfkb1, Tgfbr1, Tgfbr2, Tgfb1, and Dig4 increases a level of experience-dependent plasticity in the subject.

Embodiment 30: The method of embodiment 29, wherein the exosomes directly modulate (e.g., increase) the expression of one or a combination of genes selected from a group consisting of Calb1, Grin2b, Itpka, Neurod6, Panx2, Msi1, Cck, Nrgn, Caln1, Thy1, Marcksi1, Ncs1, Ctbp2, Git2, Nfkb1, Tgfbr1, Tgfbr2, Tgfb1, and Dig4.

Embodiment 31: The method of embodiment 29, wherein the exosomes indirectly modulate (e.g., increase) the expression of one or a combination of genes selected from a group consisting of Calb1, Grin2b, Itpka, Neurod6, Panx2, Msi1, Cck, Nrgn, Caln1, Thy1, Marcksi1, Ncs1, Ctbp2, Git2, Nfkb1, Tgfbr1, Tgfbr2, Tgfb1, and Dig4.

Embodiment 32: The method on any one of embodiments 25-31, wherein the population of neuronal cells comprises excitatory neurons, inhibitory neurons, or a combination thereof.

Embodiment 33: The method of any one of embodiments 25-32 further comprising measuring the expression of one or a combination of genes selected from a group consisting of Calb1, Grin2b, Itpka, Neurod6, Panx2, Msi1, Cck, Nrgn, Caln1, Thy1, Marcksi1, Ncs1, Ctbp2, Git2, Nfkb1, Tgfbr1, Tgfbr2, Tgfb1, and Dig4 in the population of neuronal cells prior to the administration of the exosomes.

Embodiment 34: A method of increasing neural plasticity in a subject in need thereof, the method comprising: administering nanoparticles to the subject, said nanoparticles configured to modulate (increase) the expression of calbindin (Calb1) in a population of neuronal cells in the subject, wherein modulating (increasing) the expression of Calb1 increases a level of experience-dependent plasticity in the subject.

Embodiment 35: The method of embodiment 34, wherein the nanoparticles directly modulate (increase) the expression of Calb1.

Embodiment 36: The method of embodiment 34, wherein the nanoparticles indirectly modulate (increase) the expression of Calb1.

Embodiment 37: The method of any one of embodiments 34-36, wherein the nanoparticles further modulates the expression of one or a combination of genes selected from a group consisting of Grin2b, Itpka, Neurod6, Panx2, Msi1, Cck, Nrgn, Caln1, Thy1, Marcks1, Ncs1, Ctbp2, Git2, Nfkb1, Tgfbr1, Tgfbr2, Tgfb1, and Dlg4.

Embodiment 38: A method of increasing neural plasticity in a subject in need thereof, the method comprising: administering nanoparticles to the subject, said nanoparticles configured to modulate (increase) the expression of one or a combination of genes selected from a group consisting of Calb1, Grin2b, Itpka, Neurod6, Panx2, Msi1, Cck, Nrgn, Caln1, Thy1, Marcksl1, Ncs1, Ctbp2, Git2, Nfkb1, Tgfbr1, Tgfbr2, Tgfb1, and Dig4 in a population of neuronal cells in the subject, wherein modulating (increasing) the expression of one or a combination of genes selected from a group consisting of Calb1, Grin2b, Itpka, Neurod6, Panx2, Msi1, Cck, Nrgn, Caln1, Thy1, Marcksl1, Ncs1, Ctbp2, Git2, Nfkb1, Tgfbr1, Tgfbr2, Tgfb1, and Dig4 increases a level of experience-dependent plasticity in the subject.

Embodiment 39: The method of embodiment 38, wherein the nanoparticles directly modulate (increase) the expression of one or a combination of genes selected from a group consisting of Calb1, Grin2b, Itpka, Neurod6, Panx2, Msi1, Cck, Nrgn, Cain1, Thy1, Marcksl1, Ncs1, Ctbp2, Git2, Nfkb1, Tgfbr1, Tgfbr2, Tgfb1, and Dig4.

Embodiment 40: The method of embodiment 38, wherein the nanoparticles Indirectly modulate (increase) the expression of one or a combination of genes selected from a group consisting of Calb1, Grin2b, Itpka, Neurod6, Panx2, Msi1, Cck, Nrgn, Caln1, Thy1, Marcksl1, Nos1, Ctbp2, Git2. Nfkb1, Tgfbr1, Tgfbr2, Tgfb1, and Dig4.

Embodiment 41: The method on any one of embodiments 34-40, wherein the population of neuronal cells comprises excitatory neurons, inhibitory neurons, or a combination thereof.

Embodiment 42: The method of any one of embodiments 34-41 further comprising measuring the expression of one or a combination of genes selected from a group consisting of Calb1, Grin2b, Itpka, Neurod6, Panx2, Msi1, Cck, Nrgn, Cain1, Thy1, Marcksi1, Nos1, Ctbp2, Git2, Nfkb1, Tgfbr1, Tgfbr2, Tgfb1, and Dig4 in the population of neuronal cells prior to the administration of the nanoparticles.

Embodiment 43: A method of increasing neural plasticity in a subject in need thereof, the method comprising: increasing expression of calbindin in a population of neuronal cells in the subject, wherein increasing the expression of calbindin increases neural plasticity in the subject.

Embodiment 44: A method of increasing neural plasticity in a subject in need thereof, the method comprising: increasing expression of one or a combination of genes selected from a group consisting of Calb1, Grin2b, Itpka, Neurod6, Panx2, Msi1, Cck, Nrgn, Caln1, Thy1, Marcksl1, Ncs1, Ctbp2, Git2, Nfkb1, Tgfbr1, Tgfbr2, Tgfb1, and Dig4 in a population of neuronal cells in the subject, wherein increasing the expression of one or a combination of genes selected from a group consisting of Calb1, Grin2b, Itpka, Neurod6, Panx2, Msi1, Cck, Nrgn, Caln1, Thy1, Marcks/1, Ncs1, Ctbp2, Git2, Nfkb1, Tgfbr1, Tgfbr2, Tgfb1, and Dig4 increases neural plasticity in the subject.

Embodiment 45: A method of increasing neural plasticity in a subject in need thereof, the method comprising: increasing expression of one or a combination of genes selected from a group consisting of Grin2b, Itpka, Neurod6, Panx2, Msi1, Cck, Nrgn, Cain1, Thy1, Marcksi1, Ncs1, Ctbp2, Git2, Nfkb1, Tgfbr1, Tgfbr2, Tgfb1, and Dig4 in a population of neuronal cells in the subject, wherein increasing the expression of one or a combination of genes selected from a group consisting of Grin2b, Itpka, Neurod6, Panx2, Msi1, Cck, Nrgn, Caln1, Thy1, Marcksi1, Ncs1, Clbp2, Git2, Nfkb1, Tgfbr1, Tgfbr2, Tgfb1, and Dig4 increases neural plasticity in the subject.

Embodiment 46: The method of any one of embodiments 43-45, wherein the increasing the expression of calbindin increases neural plasticity in the population of neuronal cells in the subject.

Embodiment 47: A method of increasing neural plasticity in a subject in need thereof, the method comprising: increasing expression of calbindin in a population of neuronal cells in the subject, wherein increasing the expression of calbindin increases neural plasticity in the population of neuronal cells in the subject.

Embodiment 48: A method of increasing neural plasticity in a subject in need thereof, the method comprising: increasing expression of one or a combination of genes selected from a group consisting of Calb1, Grin2b, Itpka, Neurod6, Panx2, Msi1, Cck, Nrgn, Caln1, Thy1, Marcks11, Ncs1, Ctbp2, Git2, Nfkb1, Tgfbr1, Tgfbr2, Tgfb1, and Dig4 in a population of neuronal cells in the subject, wherein increasing the expression of one or a combination of genes selected from a group consisting of Calb1, Grin2b, Itpka, Neurod6, Panx2, Msi1, Cck, Nrgn, Caln1, Thy1, Marcks11, Nos1, Ctbp2, Git2, Nfkb1, Tgfbr1, Tgfbr2, Tgfb1, and Dlg4 increases neural plasticity in the population of neuronal cells in the subject.

Embodiment 49: A method of increasing neural plasticity in a subject in need thereof, the method comprising: increasing expression of one or a combination of genes selected from a group consisting of Grin2b, Itpka, Neurod6, Panx2, Msi1, Cck, Nrgn, Cain1, Thy1, Marcksi1, Ncs1, Ctbp2, Git2, Nfkb1, Tgfbr1, Tgfbr2, Tgfb1, and Dig4 in a population of neuronal cells in the subject, wherein increasing the expression of one or a combination of genes selected from a group consisting of Grin2b, Itpka, Neurod6, Panx2, Msi1, Cck, Nrgn, Caln1, Thy1, Marcksi1, Ncs1, Ctbp2, Git2, Nfkb1, Tgfbr1, Tgfbr2, Tgfb1, and Dig4 increases neural plasticity in the population of neuronal cells in the subject.

Embodiment 50: The method of any one of embodiments 43-49, wherein the increasing the expression of calbindin increases experience-dependent plasticity in the population of neuronal cells in the subject.

Embodiment 51: A method of increasing neural plasticity in a subject in need thereof, the method comprising: increasing expression of calbindin in a population of neuronal cells in a subject, wherein increasing the expression of calbindin increases a level of experience-dependent plasticity in the subject.

Embodiment 52: A method of increasing neural plasticity in a subject in need thereof, the method comprising: increasing expression of one or a combination of genes selected from a group consisting of Calb1, Grin2b, Itpka, Neurod6, Panx2, Msi1, Cck, Nrgn, Caln1, Thy1, Marcksl1, Nos1, Ctbp2, Git2, Nfkb1, Tgfbr1, Tgfbr2, Tgfb1, and Dig4 in a population of neuronal cells in a subject, wherein increasing the expression of one or a combination of genes selected from a group consisting of Calb1, Grin2b, Itpka, Neurod6, Panx2, Msi1, Cck, Nrgn, Caln1, Thy1, Marcks/1, Ncs1, Ctbp2, Git2, Nfkb1, Tgfbr1, Tgfbr2, Tgfb1, and Dig4 increases a level of experience-dependent plasticity in the subject.

Embodiment 53: A method of increasing neural plasticity in a subject in need thereof, the method comprising: increasing expression of one or a combination of genes selected from a group consisting of Grin2b, Itpka, Neurod6, Panx2, Msi1, Cck, Nrgn, Caln1, Thy1, Marcksi1, Ncs1, Ctbp2, Git2, Nfkb1, Tgfbr1, Tgfbr2, Tgfb1, and Dig4 in a population of neuronal cells in a subject, wherein increasing the expression of one or a combination of genes selected from a group consisting of Grin2b, Itpka, Neurod6, Panx2. Msi1, Cck, Nrgn, Caln1, Thy1, Marcksi1, Ncs1, Ctbp2, Git2, Nfkb1, Tgfbr1, Tgfbr2, Tgfb1, and Dig4 increases a level of experience-dependent plasticity in the subject.

Embodiment 54: The method of any one of embodiments 43-53, wherein the population of neuronal cells comprises excitatory neurons, inhibitory neurons, or a combination thereof.

Embodiment 55: The method of any one of embodiments 43-54, wherein increasing the expression of calbindin or one or a combination of genes selected from a group consisting of Grin2b, Itpka, Neurod6, Panx2, Msi1, Cck, Nrgn, Caln1, Thy1, Marcksi1, Ncs1, Ctbp2, Git2, Nfkb1, Tgfbr1, Tgfbr2, Tgfb1, and Dig4 comprises administering one or more gene therapy agents to the population of neuronal cells in the subject.

Embodiment 56: The method of any one of embodiments 43-55, wherein the one or more gene therapy agents comprise viral vectors, exosomes, nanoparticles, small interfering RNA (SIRNA) and microRNA (miRNA), antisense oligonucleotide (ASO), aptamers, modified cells, or the like.

Embodiment 57: The method of embodiment 55 or embodiment 56, wherein the gene therapy agent directly increases the expression of calbindin or one or a combination of genes selected from a group consisting of Grin2b, Itpka, Neurod6, Panx2, Msi1, Cck, Nrgn, Caln1, Thy1, Marcksl1, Ncs1, Ctbp2, Git2, Nfkb1, Tgfbr1, Tgfbr2, Tgfb1, and Dig4.

Embodiment 58: The method of embodiment 55 or embodiment 56, wherein the gene therapy agent indirectly increases the expression of calbindin or one or a combination of genes selected from a group consisting of Grin2b, Itpka, Neurod6, Panx2, Msi1, Cck, Nrgn, Caln1, Thy1, Marcksl1, Ncs1, Ctbp2, Git2, Nfkb1, Tgfbr1, Tgfbr2, Tgfb1, and Dig4.

Embodiment 59: A method of screening for experience-dependent plasticity-inducing factors and agents, the method comprising: a) obtaining or having obtained a sample; b) administering one or more factors and agents to the sample; and c) measuring a level expression of calbindin; wherein an increase in the expression of calbindin indicates the one or more factors and agents induce experience-dependent plasticity.

Embodiment 60: The method of embodiment 59, wherein the sample is a biological sample.

Embodiment 61: The method of embodiment 59 or embodiment 60, wherein the sample comprises ex vivo tissue, primary cultures, IPSCs, organoids, or the like.

Embodiment 62: A method of screening for experience-dependent plasticity-inducing factors and agents, the method comprising: a) administering one or more factors and agents to a subject; and b) measuring a level expression of calbindin, wherein an increase in the expression of calbindin indicates the one or more factors and agents induce experience-dependent plasticity.

Embodiment 63: The method of embodiment 62, wherein the subject is an animal model (e.g., a suitable animal model).

Embodiment 64: The method of any one of embodiments 59-63 further comprising measuring the level of expression of calbindin prior to administering the one or more factors and agents.

Embodiment 65: A method of increasing neural plasticity in a subject in need thereof, the method comprising: transplanting a population of medial ganglionic eminence (MGE) cells into the subject's neuronal tissue, wherein said transplantation increases the expression of calbindin in a population of the subject's neuronal cells, wherein increasing the expression of calbindin increases a level of experience-dependent plasticity in the subject.

Embodiment 66: A method of increasing neural plasticity in a subject in need thereof, the method comprising increasing the expression of at least one of the following genes: calbindin, Grin2b, Itpka, Neurod6, Panx2, Msi1, Cck, Nrgn, Caln1, Thy1, Marcks11, Ncs1, Ctbp2, Git2, Nfkb1, Tgfbr1, Tgfbr2, Tgfb1, and Dig4, wherein the genes are overexpressed in at least one of cortical layer IV, V, or VI.

Embodiment 67: A method of increasing neural plasticity in the central nervous system of an adult subject in need thereof, the method comprising: transplanting a population of medial ganglionic eminence (MGE) GABAergic interneurons into the subject's neuronal tissue, wherein said transplantation increases the expression of calbindin in a population of the subject's neuronal cells and changes the expression of at least one of the following genes: Grin2b, Itpka, Neurod6, Panx2, Msi1, Cck, Nrgn, Cain1, Thy1, Marcksl1, Ncs1, Ctbp2, Git2, Nfkb1, Tgfbr1, Tgfbr2, Tgfb1, and Dig4, wherein increasing the expression of calbindin and at least one of Grin2b, Itpka, Neurod6, Panx2, Msi1, Cck, Nrgn, Caln1, Thy1, Marcksl1, Ncs1, Ctbp2, Git2, Nfkb1, Tgfbr1, Tgfbr2, Tgfb1, and Dig4 increases experience-dependent circuit plasticity in the adult subject.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

1. A method of increasing neural plasticity in a subject in need thereof, the method comprising: administering a gene therapy agent to the subject, said gene therapy agent configured to modulate the expression of calbindin (Calb1) in a population of neuronal cells in the subject, wherein modulating the expression of Calb1 increases neural plasticity in the subject.

2. (canceled)

3. The method of claim 1, wherein modulating the expression of Calb1 increases experience-dependent plasticity in the subject.

4. The method of claim 1, wherein the gene therapy agent increases the expression of Calb1.

5. The method of claim 1, wherein the population of neuronal cells comprises excitatory neurons, inhibitory neurons, or a combination thereof.

6. The method of claim 1, wherein the gene therapy agent comprises viral vectors, exosomes, nanoparticles, small interfering RNAs (siRNAs) and microRNAs (miRNAs), antisense oligonucleotides (ASOs), aptamers, modified cells, or the like.

7. The method of claim 1, any wherein the gene therapy agent directly increases the expression of Calb1.

8. The method of claim 1, wherein the gene therapy agent indirectly increases the expression of Calb1.

9. The method of claim 1, further comprising measuring the expression of Calb1 prior to administering the gene therapy agent.

10. The method of claim 1, wherein the gene therapy agent compirses a viral vector configured to increase the expression of Calb1.

11-27. (canceled)

28. A method of increasing experience-dependent plasticity in a subject in need thereof, the method comprising: increasing expression of calbindin (Calb1) in a population of neuronal cells in the subject, wherein increasing the expression of Calb1 increases neural plasticity in the subject.

29. The method of claim 28, wherein increasing the expression of Calb1 increases a level of experience-dependent plasticity in the subject.

30. The method of claim 28, wherein the population of neuronal cells comprises excitatory neurons, inhibitory neurons, or a combination thereof.

31. The method of claim 28, wherein increasing the expression of Calb1 comprises administering one or more gene therapy agents to the population of neuronal cells in the subject.

32. The method of claim 31, any wherein the one or more gene therapy agents comprise viral vectors, exosomes, nanoparticles, small interfering RNA (siRNA) and microRNA (miRNA), antisense oligonucleotide (ASO), aptamers, modified cells, or the like.

33. The method of claim 31, or wherein the gene therapy agent directly increases the expression of Calb1.

34. The method of claim 31 wherein the gene therapy agent indirectly increases the expression of Calb 1.

35. A method of screening for experience-dependent plasticity-inducing factors and agents, the method comprising:
a) administering one or more factors and agents to a sample; and
b) measuring a level expression of calbindin wherein an increase in the expression of calbindin indicates the one or more factors and agents induce experience-dependent plasticity.

36. The method of claim 35 further comprising obtaining or having obtained a sample.

37. (canceled)

38. The method of claim 35, wherein the sample is a biological sample.

39. The method of claim 35, wherein the sample comprises ex vivo tissue, primary cultures, iPSCs, organoids, or the like

40-42. (canceled)
